(19) United States
(12) Patent Application Publication
(10) Pub. No.: US 2023/0089826 A1
(43) Pub. Date: Mar. 23, 2023

(54) TRANS-ANAL INFLOW CATHETER FOR INTERMITTENTLY TRIGGERING A REFLEX-COORDINATED DEFECATION

(71) Applicant: Advanced Medical Balloons GmbH, Waghäusel (DE)

(72) Inventor: Fred Göbel, Speyer (DE)

(21) Appl. No.: 17/993,340

(22) Filed: Nov. 23, 2022

Related U.S. Application Data
(63) Continuation-in-part of application No. 16/026,873, filed on Jul. 3, 2018, now abandoned.
Application No. 16/026,873 is a continuation-in-part of application No. 14/239,991, filed on Jun. 13, 2014, now abandoned.

(30) Foreign Application Priority Data
Aug. 20, 2011 (DE) 10 2011 111 225.5
Nov. 21, 2011 (DE) 10 2011 118 943.6
Dec. 16, 2011 (DE) 10 2011 121 202.0
Mar. 22, 2012 (DE) 10 2012 005 607.9
Apr. 25, 2012 (DE) 10 2012 008 361.0

(51) Int. Cl. A61M 25/10; A61M 3/02

(52) U.S. Cl. CPC A61M 25/1002; A61M 3/0295

(57) ABSTRACT

A device for intermittently triggering a coordinated defecation reflex in the rectum or colon of a patient, said device comprising a catheter with an inflexible catheter shaft and a preformed inflatable balloon, the inflatable balloon comprising at least an intrarectal balloon segment having, in an inflated, but unpressurized state of the balloon, a generally spherical or discoid shape and a maximal first radius, and being adapted to be placed intrarectally; wherein the inflatable balloon is fixed onto the catheter shaft only at fixation points located at the ends of the inflatable balloon, wherein both ends of the inflatable balloon taper to a shaft dimension of the catheter shaft supporting the inflatable balloon, with the distal end of the balloon having a tapered second radius; wherein, in an inflated, but unpressurized state of the balloon, the difference between the first radius of the intrarectal balloon segment and the second radius of the distal tapered balloon end is equal to a first difference, and further wherein the first difference is greater than a distance between a distal front side of the catheter and a distal fixation point, where the tapered distal end of the balloon is in contact with the catheter shaft and is fixed thereto; wherein the distal front side of the catheter is at a distalmost extension of the catheter shaft or of a distal tip piece of the catheter shaft; and wherein the inflatable balloon is configured such that when the inflatable balloon is in a pressurized state exceeding 30 mbar, at least a portion of the distal intrarectal balloon segment engages and expands a side wall of the rectum or colon of the patient, whereby to trigger a coordinated defecation reflex in the rectum or colon of the patient.

### REFERENCE TO PENDING PRIOR PATENT APPLICATIONS

This patent application is a continuation-in-part of pending prior U.S. patent application Ser. No. 16/026,873, filed Jul. 3, 2018 by Fred Göbel for TRANS-ANAL INFLOW CATHETER FOR INTERMITTENTLY TRIGGERING A REFLEX-COORDINATED DEFECATION (Attorney's Docket No. KUCH-59 CIP 1), which patent application, in turn, is a continuation-in-part of prior U.S. patent application Ser. No. 14/239,991, filed Feb. 20, 2014 by Fred Göbel for TRANS-ANAL INFLOW CATHETER AND METHOD FOR INTERMITTENTLY TRIGGERING A REFLEX-COORDINATED DEFECATION (Attorney's Docket No. KUCH-59), which patent application, in turn, is a 371 national stage entry of prior International (PCT) Patent Application No. PCT/EP2012/003535, filed Aug. 20, 2012 by Fred Göbel for TRANS-ANAL INFLOW CATHETER AND METHOD FOR INTERMITTENTLY TRIGGERING A REFLEX-COORDINATED DEFECATION, which patent application in turn: (i) claims benefit of prior German Patent Application No. 10 2011 111 225.5, filed Aug. 20, 2011; (ii) claims benefit of prior German Patent Application No. 10 2011 118 943.6, filed Nov. 21, 2011; (iii) claims benefit of prior German Patent Application No. 10 2011 121 202.0, filed Dec. 16, 2011; (iv) claims benefit of prior German Patent Application No. 10 2012 005 607.9, filed Mar. 22, 2012; and (v) claims benefit of prior German Patent Application No. 10 2012 008 361.0, filed Apr. 25, 2012.

The eight (8) above-identified patent applications are hereby incorporated by reference.

### FIELD OF THE INVENTION

The invention relates to a device for transanally introducing an infusion into the rectum or colon of a patient, and/or for intermittently triggering a coordinated defecation reflex in the rectum or colon of a patient, said device comprising a catheter with an inflexible catheter shaft and a preformed inflatable balloon, the inflatable balloon comprising at least an intrarectal balloon segment having, in an inflated, but unpressurized state of the balloon, a generally spherical or discoid shape and a maximal first radius, and which is adapted to be placed intrarectally, wherein the inflatable balloon is fixed onto the catheter shaft only at fixation points located at the ends of the inflatable balloon, wherein both ends of the inflatable balloon taper to a shaft dimension of the catheter shaft supporting the inflatable balloon, with the distal end of the balloon having a tapered second radius.

The wording “inflexible catheter shaft” shall describe a behaviour which is sufficiently rigid in order to allow a self-insertion of the catheter shaft into the anus and rectum of a patient. Especially, in case of an axial thrust force, the catheter shaft should resist any kinking deformation. Furthermore, it should be radially stable, i.e. not allowing for a collapse of the catheter lumen. Additionally, the catheter shaft should resist any twisting or torsion movement. On the other hand, in order to avoid lesions of the bowel wall, the catheter shaft may have a minor ability of an axial bending, but such axial bending ability should be limited, for example to a bending angle of not more than 45° over the entire length of the catheter shaft.

In the context of this patent application, the wording “an intrarectal balloon segment having . . . a generally spherical or discoid shape” shall be understood as being no substantial toroidal shape, at least along the length, where the intrarectal balloon segment extends along the catheter shaft. In other words, the tapered balloon section adjacent to the distal balloon end which is fixed to the catheter shaft shall not extend between the shaft and the radially outer portion of the intrarectal balloon segment. Of course, the spherical or discoid intrarectal balloon segment is penetrated by an open central lumen, but this central lumen shall be defined by the catheter shaft only, where possible. The reason is that a rolling or movement of the balloon in an axial direction is undesired. The primary deformation of the intrarectal balloon segment shall be restricted to a radial direction.

Another feature of this geometry of the balloon according to the invention is that, in a longitudinal direction of the catheter shaft, the intrarectal balloon segment shall not extend beyond both fixation points of the balloon ends to the catheter shaft. It shall not overlap the distal fixation point in the distal direction and at the same time overlap the proximal fixation point in the proximal direction.

Furthermore, the space inside the balloon element shall comprise only one single compartment.

Finally, if placed in situ, the catheter shaft extends through the anus of the patient to the outside to allow a user to grip it.

### BACKGROUND OF THE INVENTION

Catheters for transanally administering an infusion into the rectum or higher portions of the large bowel (colon) of a patient have been known in a wide variety of embodiments for many years.

Inflow catheters can be implemented as simple tube elements, but for improved retention of the catheter in the rectum they are also provided with an intrarectally placed balloon element. Such balloon elements provide, in addition to an anorectally anchoring function, a certain sealing effect that retains in the bowel the fluid that has been introduced by the irrigation. The sealing capacity of inflow catheters fitted with an exclusively intrarectal balloon component is inadequate in many cases, however, and must be created by the user by continuously manually repositioning the surfaces of the sealing balloon on the floor of the rectum or manipulating the position of the catheter shaft atop the balloon.

A major risk associated with the use of ordinary commercial catheters that are inserted transanally in the bowel continues to be perforation of the bowel wall due to improper handling or the presence of previously injured or similarly weakened bowel wall structures. Such perforations of the intestine have been preventable heretofore only by suitable education and sensitization of the user with respect to this particular set of problems.

Over the past ten years, a novel infusion technology that goes beyond the use of conventional transanal infusions has become established; in this new approach, the reflex-coordinated, active evacuation of the rectum and large portions of the left colon adjoining the rectum is triggered by intermittent, relatively small-volume, intrarectally introduced infusions. By consistent voiding of these segments of the intestine, the patient can thus be brought to a state of so-called “pseudo-continence” regardless of existing incontinence problems. The so-called transanal irrigation (TAI) method can be performed by the patient himself.

The function of the rectally instilled infusion medium, apart from some degree of mobilization of stool, is primarily to moderately expand the wall portions of the rectal ampulla (the terminal rectum), which then results in the triggering of the physiological stool evacuation reflex (reflex triggering). Whereas the rectally placed infusion medium usually causes such reflex triggering within a few minutes, it can take much longer, up to 30 minutes or more, for the administered irrigation volume to be fully voided from the rectum or colon. The relatively long time required for TAI limits its acceptance by many patients, and often rules this method out despite the fact that it is, as a rule, efficient for use by the individual.

Another problem is that many users are not able to manually bring irrigation catheters of conventional design into an adequately sealing position and keep them there.

Furthermore, as the balloon component of a conventional catheter is expanded inside the rectum, a reflex-triggering expansion of portions of the bowel wall can occur when the filling process has only just begun, thereby leading, inter alia, to a reflex-like opening of the anus and thus sometimes causing the anchoring balloon to slip out of the rectum.

Especially when using air as a filling medium for expanding a conventional retention balloon, standard high-compliant polymeric materials allow for uncontrolled, unlimited volume expansion, potentially causing a trauma risk to the patient.

Furthermore, commercially available devices for intermittent transanal irrigation, such as, for example, Peristeen Anal Irrigation, made by Coloplast, Denmark, are of relatively elaborate design, and are often only conditionally suitable for use outside the patient's familiar domestic surroundings.

To improve user acceptance of transanal irrigation, a catheter technique would be desirable which, once the catheter shaft is inserted in the rectum, holds it in its transanal position without the need for continuous repositioning, and both keeps the body of the shaft from sliding on into the bowel, with potentially traumatic consequences, and reliably prevents the catheter from accidentally slipping out of the anus prematurely.

Greater convenience in terms of the transanal placement of the catheter is offered in this regard by inflow catheters having a double balloon arrangement. Here, two separate balloon elements, which are usually filled simultaneously via a single filling lumen, are mounted in spaced relation on the catheter shaft. These come to lie on both sides of the anal canal, i.e. the internal and the external, thus mutually taking on the structures of the anus. When the wall of the balloon is expanded under pressure, the usually elastic implementation of the balloon material brings about a spherical enlargement of the balloon bodies and thus a certain axially exerted squeezing of the anus disposed between the balloon bodies. The dislocation-proof positioning of the catheter shaft obtained with such catheters can be assumed to be relatively reliable. A problem resides in the sometimes high filling pressures needed for the elastic expansion of the envelope, which are felt by patients as an uncomfortable or even painful foreign body sensation. Further, the elastic expansion of the intrarectal balloon to a tightly filled sphere can lead to direct triggering of the defecation reflex, which, in turn, in the presence of simultaneous rectal contraction and declining sphincter muscle tone, will in the most unfavorable case cause the catheter to slide out and the irrigation fluid to be voided prematurely.

DE 10 2004 033 425 B4 as well as US 2007/0 213 661 A1 describe a sealing system for the management of rectal or anal incontinence having a particular embodiment for the tamponade of bleeding hemorrhoids, presenting a waisted balloon element having a terminal intrarectal or preanal section. The distal end of the intrarectal balloon segment is reverse folded and extends into the radially outer portion of the intrarectal balloon segment like a torus, and therefore, the intrarectal balloon segment has no generally spherical or discoid shape. A disadvantage of such a toroidal geometry is that the balloon tends to an axial rolling movement in case of an expansion of the balloon towards its working dimension or beyond, instead of a primary growth in the radial direction. The balloon envelope of the device is placed on the balloon-supporting shaft body in such a way that during the filling process, the intrarectal balloon segment moves in the direction towards the rectal floor and there exerts a tamponade pressure on the bleeding venous blood vessels. The preanal balloon segment concurrently moves toward the external anal opening. The result is a tamponade effect oriented axially to the anus from both sides. In addition to the axial tamponade of the bleeding, the middle, waisted section of the balloon expands outward radially to the wall of the anal canal. It is preferably shaped with a diameter that exceeds the diameter of the opened anal canal. In the described device for the acute management of venous anorectal bleeding, in the in-use state the tip of the shaft body protrudes freely and unprotectedly into the intestinal lumen and poses a potential risk of injury.

The document WO 2009/144 028 A1 discloses a device for sealing a natural or artificial opening in the colon. In most embodiments of this document, one or both ends of the balloon are reverse folded into a radially outer portion of the intrarectal balloon segment providing a toroidal structure, and are forming a flexible shaft, instead of being fixed to an inflexible catheter shaft and/or form a toroidal structure instead of a spherical or discoid structure. Other embodiments disclosed in this document comprise more than one single compartment, for example an intrarectal compartment and another compartment which is to be placed trans-anally. It should also be noted that this document deals with a device for a long-term drainage, which can remain in a patient for days or even weeks. For such an application, it is necessary that a shaft surrounding a central lumen is flexible and causes no traumata, whereas in the present invention, a triggering of a coordinated defecation reflex in the rectum or colon of a patient last only several minutes, until the defecation reflex is initiated. Then, the device is removed from the colon, either by a manual operation of the user or by the defecation reflex itself which attempts to clear the colon from all contents, even from the device, too. In such an application, another feature is more important than a flexible shaft, namely a proper handling, and in this view, an inflexible catheter shaft improves the insertion procedure into the anus much better than a flexible shaft.

U.S. Pat. No. 3,459,175 B1 discloses a medical device for control of enemata, comprising a hollow pipe adapted to be inserted by its forward end through the anal canal into the lower region of a patient's bowel and being adapted to connect by its rear end with an enema tube, wherein an inflatable annular element of resilient flexible material is fixed upon said hollow pipe. Here, in an inflated, but unpressurized state of the balloon, the difference between the outer radius of an intrarectal balloon segment and a tapered balloon section adjacent to the distal end of the balloon is smaller than the distance between a distal fixation point of the balloon and the distal front side of the catheter balloon. Furthermore, the balloon element is only used to anchor the distal end of the device in the rectum of a patient, but cannot be expanded sufficiently in order to get in contact with trigger-sensitive areas at the walls of the rectum or colon of a patient.

WO 2007/118 621 A1 describes a dumbbell- or hourglass-shaped balloon sealing system as a surrogate for a tamponade. The object there is to provide a seal against involuntary leakage of stool for intermittent periods of a few hours in chronically anorectally incontinent patients. This managing device also prominently features the rolling movement of the terminal balloon segments from both sides axially toward the anus, instead of a primary movement in the radial direction. Here again, in the transanally placed state, the tip of the shaft body supporting the balloon protrudes freely and potentially traumatizingly into the intestinal lumen and in order to avoid injuries, the shaft body has to be formed as an elastically deformable plastic tube, which presents problems upon insertion into the anus. Furthermore, according to this document, the distal end of the intrarectal balloon segment is invaginated into the radially outer portion of the intrarectal balloon segment, and therefore, the intrarectal balloon segment has no generally spherical or discoid shape, too.

Both documents DE 10 2004 033 425 B4 as well as WO 2007/118621 A1 describe devices with an an hourglass- or dumbbell-shaped balloon configuration, the anus being received in the waisted region of the balloon, i.e. the region that is tapered relative to the terminal portions thereof. Such shaping of the balloon not only results in relatively good securing of the catheter against dislocation, due to the sealing of the balloon inside the anal canal on all sides, but also makes for a considerable improvement in sealing performance over that of conventional double balloons. In addition, both devices make it possible to largely avoid any expansion of the balloon envelope that could trigger irritation, pain or a defecation reflex, since the balloon wall is preferably already shaped to its working dimensions or beyond. Thus, the filling or unfolding of the balloon to its working state requires only a low filling pressure, equal to or only slightly greater than the pressure prevailing in the rectum or abdomen at the time.

It has been shown in clinical use on patients, that an hour-glass shaped balloon, having a waisted, middle segment for enhanced trans-anal positioning of the catheter device, can be associated with a certain “volume steal effect”, as such that at an increase of filling pressure inside the balloon, the preanal segment of the balloon, which unfolds freely outside the body, other than the intrarectal balloon segment, being exposed to an expansion limiting, counteracting resistance of the rectal tissue and/or the actual recto-abdominal force, expands in diameter to a degree, that the entire balloon is potentially luxated out of its trans-anal position and out of the body.

It has also proven important for the optimal performance of balloons with a tapered transanal segment, that the transanal segment is not, or only little increasing in diameter from the pre-shaped dimension, within the pressure range typically applied for dilation of the rectal wall. On the other hand, it has been shown beneficial when the intra-rectal segment is equipped with a certain compliance, allowing for a certain, dimensionally limited expansion of the intra-rectal balloon portion beyond its pre-shaped measures.

When using highly compliant materials for rectally positioned balloons, as e.g. silicone or rubber based polymers, these components are associated with the risk of unlimited expansion, which may result in uncontrolled organ dilation and trauma. Further, the over-inflated balloon envelope may rupture and decompress the filling medium. The balloon component shall therefore perform a self-limitating expansion, which can be granted by use of low-compliant, highly elastic materials, as e.g. polyurethanes, also allowing for pre-forming of balloon bodies to pressure stable, sustaining shapes.

Another disadvantage of the embodiments described in DE 10 2004 033 425 B4 and WO 2007/118621 A1 has proven to be that the distal tip of the shaft body supporting the balloon becomes freely exposed during the filling process and protrudes into the intestinal lumen, potentially causing irritation or injury to the bowel wall there during use.

US 2010/0 087 792 A1 suggests a switching device as an extracorporeal part of an irrigation system. This switching device comprises a housing and a grippable portion of a selection handle, a balloon pump, which—upon manual action—provides regular air under pressure which can be supplied via a port to a catheter connected thereto. Details of the catheter itself are not available from this document.

U.S. Pat. No. 7,122,025 B1 presents a rectal insertion device for the treatment of disorders of the digestive tract of a human or animal patient, having a body with a central lumen, but without any balloon. As this device does not comprise any expandable element, it cannot be used to trigger a defecation reflex by a radial expansion.

US 2009/0 171 278 A1 refers to a balloon catheter system for treating vascular occlusions. Such catheter is inserted into a blood vessel by use of a guide wire, and therefore cannot be used to trigger a defecation reflex in the colon of a patient. Furthermore, the distal fixation point has a larger distance from the front side of the catheter shaft than from the outer radius of the balloon.

### SUMMARY OF THE INVENTION

The problems initiating the invention is to eliminate these disadvantages of the known prior art as explained above.

This problem is solved at a generic device in that, in an inflated, but unpressurized state of the balloon, the difference between the first radius of the intrarectal balloon segment and the second radius of the distal tapered balloon end is equal to a first difference, and further wherein the first difference is greater than a distance between a distal front side of the catheter and a distal fixation point, where the tapered distal end of the balloon is in contact with the catheter shaft and is fixed thereto; wherein the distal front side of the catheter is at a distalmost extension of the catheter shaft or of a distal tip piece of the catheter shaft; and wherein the inflatable balloon is configured such that when the inflatable balloon is in a pressurized state exceeding 30 mbar, at least a portion of the distal intrarectal balloon segment engages and expands a side wall of the rectum or colon of the patient, whereby to trigger a coordinated defecation reflex in the rectum or colon of the patient.

As the distal front side of the catheter is defined by a distalmost extension of the catheter shaft or of a distal tip piece of the catheter shaft, always the portion of the catheter projecting maximally into the rectum of the patient has to be applied by measuring the difference between the distal fixation point and the distal front side of the catheter.

Preforming the balloon to its working dimension further makes it possible for the user, by increasing the filling pressure or fill volume of the balloon from the initial filled state in which the balloon primarily has a sealing and anchoring effect, to induce a well-controllable and gradually increasable expansion of the bowel wall adjacent the catheter balloon, ultimately leading to controlled triggering of the defecation reflex with individually adjusted intensity.

In case of need, the intensity of such pneumatic expansion of the bowel wall can substantially exceed the intensity of reflex-triggering expansion with fluid media. Due to the more intense stimulus, several sequentially occurring cycles of evacuation reflexes can be triggered even though the stimulation is given only once.

This reflex-triggering expansion of at least a distal intrarectal balloon segment is rather independent from the form of the balloon proximally adjacent to the distal intrarectal balloon segment.

In a first, most simple manifestation, the balloon may taper to the dimension of the catheter shaft immediately proximally adjacent to the intrarectal balloon segment, and can be fixed to the catheter shaft at the proximal fixation point. In this case, the balloon does not substantially extend into the trans-anal passage of the patient. In the pending patent application, this embodiment will be characterized by an overall sperical or discoid-shaped balloon, disregarding the central lumen provided by the catheter shaft penetrating the balloon.

The above-mentioned arrangement can be improved further in that, proximally to the intrarectal balloon segment, the balloon may taper halfway or partially, that is only to a dimension greater than the catheter shaft, and this partially tapered balloon segment may extend into the trans-anal passage of the patient, providing an improved sealing effect within the anal canal. Outside of the anal-canal, the tapered balloon segment may taper further to the dimension of the catheter shaft, and can be fixed to the catheter shaft at the proximal fixation point. In the pending patent application, this embodiment will be characterized by a mushroom-shaped balloon, disregarding the central lumen provided by the catheter shaft penetrating the balloon.

The preceding configuration can be improved further in that the partially tapered balloon may widen proximally to the trans-anal balloon segment, and this widened balloon segment forms a preanal balloon segment, providing an improved anchoring action of the device preventing it from slipping entirely inside of the rectum. Proximally to the widened preanal balloon segment, the balloon may taper to the dimension of the catheter shaft, and can be fixed to the catheter shaft at the proximal fixation point. In the pending patent application, this embodiment will be characterized by a dumbbell-shaped or hourglass-shaped balloon, disregarding the central lumen provided by the catheter shaft penetrating the balloon.

In all afore-mentioned manifestations, the intrarectal balloon segment has a spherical or discoid shape and is used to trigger a coordinated defecation reflex upon an expansion thereof by a balloon pressure exceeding 30 mbar. In such a pressurized state, the intrarectal balloon segment engages and expands a side wall of the rectum or colon of the patient. As the pressure exerted onto the rectum or colon of a patient is less than 100 mbar, or even less than 70 mbar, for example between 30 mbar and 50 mbar, the rectum or colon of the patient is less likely to react with an immedieate, but uncoordinated defecation reflex. Nevertheless, as the pressure persists for several minutes, a filled rectum is simulated and thereby, a coordinated defecation reflex in the rectum or colon of the patient is triggered. Different from an uncoordinated defecation reflex, which is unable to move intestinal contents from the descending colon downwards towards the rectum and eventually out of the anus, a coordinated defecation reflex is able to evacuate the left-sided, descending colon from its contents rather entirely.

If the catheter balloon comprises, additionally to the intrarectal balloon segment having the first radius, a tapered transanal balloon segment disposed proximally adjacent to the intrarectal balloon segment and having a third radius, the invention provides that the first radius of the intrarectal balloon segment is larger than the third radius of the tapered transanal balloon segment.

Furthermore, the invention provides, that (i) in the pressurized state of the inflatable balloon at 30 mbar, the difference between the first radius of the intrarectal balloon segment and a third radius of the transanal balloon section is equal to a second difference, and (ii) in the pressurized state of the inflatable balloon at 100 mbar, the difference between the first radius of the intrarectal balloon segment and the third radius of the transanal balloon section is equal to a third difference, and further wherein the second difference is less than the third difference.

Furthermore, in case of an increased pressure in the balloon beyond 100 mbar, the difference between the larger radius of the distal terminal balloon section and the reduced radius of the proximally adjacent, tapered balloon section will approximate a constant value.

The invention can be further improvid in that the tapered transanal balloon section being adapted to be placed transanally expands by less than 20%, or by less than 15%, at a pressure increase from 30 to 100 mbar.

On the other hand, it is recommended that the intrarectal balloon segment being adapted to be placed intrarectally expands by more than 15%, or by more than 20%, at a pressure increase from 30 to 100 mbar.

In order to distinguish the invention over conventional enema-based evacuation methods for the colon, which use a rectally infused liquid to mobilize and dissolve the feces and wash the intestinal contents out of the colon, the invention uses only air or another gas to fill the intrarectal balloon segment, which then triggers a coordinated defecation reflex which then evacuates the left-sided portion of the colon, the method according to the present invention shall be denoted as “pneumatic” method for triggering a defecation reflex. As by such method, the strain-sensitive cells in the wall of the rectum can be pointedly stimulated over a long term with a relative low pressure, coordinated defecation reflexes will be triggered which are able to empty the entire left-sided, descending colon, and thereby, the patient can be set in a condition of quasi continence for several hours.

Thereby, additionally to the initial expansion of the wall of the rectum, a further suitable increase in the filling pressure in the dumbbell-shaped, waisted catheter balloon additionally causes a dilation of the anus or the anal sphincter, constituting another efficient trigger stimulus. The intensity of such pneumatic expansion of the anus can substantially exceed the intensity of reflex-triggering expansion with fluid media.

So, the present invention provides two stages of trigger stimuli. The primary trigger stimulus is a radial expansion of the intrarectal balloon segment towards the lateral wall of the rectum, and a secondary trigger stimulus is an expansion of the tapered trans-anal balloon segment radially outwards in a direction towards the sphincter. On the other hand, due to a rather small pressure of about 30 mbar to 50 mbar, these stimuli are limited and take effect only after a timespan of several minutes.

The problem of “volume steal” from the intra-rectal segment to a preanal segment and a potential luxation of the balloon from its transanal position out of the body can be solved or largely limited in various manners. First, as a less preferable option, the pre-anal balloon segment can be taken out of the design, comprising a balloon, only having an intra-rectal segment and a transanal segment, whereby the transanal segment does not exceed the anal opening, ending on the level of the anal rim. Second, the trans-anal segment of the balloon extends out of the anal opening, but not having a spherical or discoidal enlargement, having a length of preferably not more than 2 cm. Third, the preanal segment can be given a conical shape, whereby the conus increases in diameter from the diameter of the transanal segment, near the anal rim, to the proximal diameter of its basis, by about 50 to 75%, and the length of the conus preferably does not exceed 2 cm.

When performing a reflex triggering stimulation by a pneumatic dilation of the rectum and the anus, the trans-anal balloon segment shall not expand in diameter beyond its pre-shaped dimension, ranging between 2.0 to 3.5 cm, being sufficient for a reflex triggering widening of the anal sphincter. A diameter increase beyond the preshaped dimension would be potentially impairing the trans-anal positioning of the device and not contributing to a better stimulation result.

The intrarectal balloon segment, on the other hand, shall have an optional compliance reserve, enabling the intrarectal segment to dilate the rectum beyond the pre-shaped diameter of the intrarectal segment, at higher balloon filling pressures, respectively in case of need, whereby the dimensional growth at elevated filling pressures is limited.

The problem of uncontrolled, unlimited expansion of the balloon envelope and the associated risk of balloon burst, is solved by the invention by using particular elastomeric polymers, limiting the elastic expansion of a balloon by the particular elasticity properties of the polymer itself. Especially polyurethanes in a durometer range of Shore of 60A to 75A, or 80A to 95A and 55D to 60D can be used within the scope of the invention. By the invention, thin-walled hour-glass shaped balloon structures are manufactured by blow-molding a pre-extruded tubing. The achieved balloon structure distends elastically from the pre-shaped dimension to a final dimension, that is not allowing for further dimensional expansion, where distension comes to a rest. Such self-limiting expansion of a balloon envelope suggests polyurethanes the ideal material for rectally positioned, pneumatically reflex triggering balloons.

Further advantages can be achieved if a wall thickness in the intrarectal balloon segment adapted to be placed intrarectally is 3 to 6 times thinner than a wall thickness in the tapered transanal balloon section adapted to be placed transanally, or is 4 to 5 times thinner than the wall thickness in the tapered transanal balloon section.

The aforementioned feature can be improved further in that a wall thickness in the intrarectal balloon segment adapted to be placed intrarectally is 10 to 30 μm, or is 15 to 25 μm.

On the other hand, a wall thickness in the tapered transanal balloon section adapted to be placed transanally can be 30 to 180 μm, or can be 60 to 125 μm.

The tapered transanal balloon section may be configured to terminate flush with the anus.

The present invention may receive a further improvement in that the length of the tapered transanal balloon section is designed such that in case of a placement of the device in a patient, the tapered transanal balloon section shall project over the anus to form a pre-anal segment, wherein the pre-anal segment of the balloon is pre-formed into a conical balloon section having a conical shape with a distal end and a proximal end, wherein a pre-formed diameter at the proximal end of the conical balloon section is 30 to 70% larger, or 40 to 60% larger, than a pre-formed diameter at the distal end of the conical balloon section.

According to the invention, it is possible that the intrarectal balloon segment adapted to be placed intrarectally and the tapered transanal balloon section adapted to be placed transanally are manufactured separately, or are manufactured from different materials and/or with different characteristics, or are manufactured from materials with different material characteristics like different durometers.

Furthermore, the tapered transanal balloon section adapted to be placed transanally can have less volume-expandable characteristics and/or less elasticity than the intrarectal balloon segment adapted to be placed intrarectally.

Preferably, the intrarectal balloon segment adapted to be placed intrarectally is made from polyurethane with a Shore hardness of 80A to 90A.

wherein the tapered transanal balloon section adapted to be placed transanally is made from polyurethane with a Shore hardness of 95A or of 55D to 60D.

If the inflatable balloon has a waisted shape, comprising the intrarectal balloon segment of the first radius and of generally spherical or discoid shape, a proximal terminal balloon section of a fourth radius and of generally spherical or discoid shape, and, disposed between them, the tapered transanal balloon section having a third radius, with the tapered balloon section of the device being adapted to be placed transanally, and the intrarectal balloon segment being adapted to be placed intrarectally, and the proximal terminal balloon section being adapted to be placed extracorporeally, then the fourth radius shall be larger than the third radius in an unfolded, but pressureless condition of the balloon.

It is preferred that the ends of the inflatable balloon, beginning at an inflection point of their longitudinal section, are inwardly invaginated, or inverted, and in the inverted or invaginated state are fixed on an outer jacket surface of the catheter shaft.

The ends of the inflatable balloon shall be fixed on an outer jacket surface of the catheter shaft such that, at a transanally placement of the balloon in situ, the balloon waist seals inside and against the anal canal in a radial direction on all sides.

Furthermore, at a transanally placement of the balloon in situ, the catheter shaft can be displaced inside the anal canal by a tug or push on a tube connection at the catheter shaft by which the catheter is guided into the anal canal.

The problem of catheter tip caused perforation of the bowel wall can be solved in two manners. First, the position of the catheter tip to the distal fixation point of the catheter balloon on the catheter shaft can be limited to the difference in diameter between the transanal and the intrarectal balloon segment, measured in the freely inflated, non pressurized state of the inflated balloon. When positioned inside the rectum, the catheter tip, in a non-distended state of the balloon, exceeds the distal balloon radius only slightly. Upon an accidental swiveling movement of the tip of the catheter, where the trans-anal segment of the catheter balloon acts as a bearing inside the anus for such movement, a small relative length of the catheter tip will result in a small lateral movement of the catheter tip, thereby minimizing the risk of injury.

Second, an atraumatic positioning of the catheter tip can be gained by virtue of the fact that both balloon ends taper to the shaft dimension of the catheter shaft supporting the balloon and are fixed in a simply invaginated or inverted manner on the preferably outer jacket surface of the catheter shaft in such fashion that as the balloon is filled, the two radially enlarged balloon sections move toward each other in opposite axial directions, and wherein the two radially enlarged balloon sections are enlarged relative to the tapered, middle balloon section, such that when the balloon is placed transanally, during the filling process the two radially enlarged balloon sections draw down over the middle, tapered balloon section and in the limit case come into direct contact with each other, thereby limiting their relative movement and preventing the distal end of the catheter shaft from passing beyond the apex of the intrarectal balloon radius when the shaft body is in a position of maximum axial deflection.

To prevent, insofar as possible, lesions due to the tip of the shaft, the invention describes a specific, particularly advantageous ratio of the length of the middle, waist-like balloon segment to the invagination (inversions), on both sides, of the fixation points of the ends of the balloon shaft to the catheter shaft. This ratio ensures that in the filled, transanally positioned state, the tip of the catheter shaft spontaneously retracts into the intrarectal balloon segment and is nestled atraumatically there. The atraumatic securing of the catheter tip inside the intrarectal balloon segment is also ensured according to the invention when the shaft axis undergoes the deflections inside the anal canal that typically occur during use, of the kind readily caused by a tug or push on the tube connection through which the catheter is guided in.

Preferably, as the balloon is filled, a distal tip of the catheter shaft, in a non-displaced resting state, retracts into a protected, injury-preventing position inside the distal terminal balloon section adapted to be placed intrarectally and, in so doing comes to lie entirely proximally of a distal apex of a circle about a center point which is located on an axis of symmetry and whose tangent in an inflection point corresponds to a tangent to a non-invaginated longitudinal section of the balloon.

It is further recommended that the distal tip of the catheter shaft in its non-deflected, resting state comes to lie entirely proximally of a plane Z which is intersected perpendicularly by an axis X of symmetry and which is fully tangent distally to the intrarectal balloon section in an inflated, but unpressurized state of the balloon.

The tapered transanal balloon section can be separated from each of the approximately spherical or discoid balloon sections by a respective inflection point of a curvature of the balloon along a longitudinal direction and in a non-expanded filled state has an axial length A that is larger than its minimum diameter.

Furthermore, the balloon according to the invention is characterized by invaginated or inverted balloon ends which, in the non-expanded filled state, each have an axial extent B₁, B₂ that is greater than their minimum diameter.

The invention recommends that, in the non-expanded filled state of the balloon, a sum of the axial extents B₁, B₂ of respective inversions of the two balloon ends are at least equal to the length of the tapered balloon section A:

B₁+B₂≥A.

In case a catheter tip projects by a dimension C past a forward fixation line of a forward balloon end on the catheter shaft, an inversion depth B shall be greater than or equal to half the length A plus the length C of the projecting tip piece:

B₁=B₂≥A/2+C,

or alternatively:

B₁=B₂≥A/2+C/2.

A preferred result is that even upon axial deflection of the catheter shaft toward the bowel in response to forces that typically occur during use, the catheter tip will not not move more than a distance W beyond an apex of the forward balloon radius, and distance W is obtained from a distance between a first apex and a second apex, wherein the second apex is defined as the forward intersection point of the axis X of symmetry with a circle of diameter D around a center point on the axis X of symmetry at a level of the attachment of the intrarectal balloon section to the catheter shaft.

The invention further addresses the problem of preventing undesired or premature triggering effects due to the filled balloon as it unfolds in the rectum. Triggering of the defecation reflex can be prevented in most cases by preshaping the balloon to the working dimension or beyond (residual dimensioning), since the filling pressures necessary for anchoring and sealing the catheter are largely the same as, or need be only a few millibars higher than, the pressures prevailing in the rectum or in the abdomen. The atraumatic securing of the tip of the catheter in the region of the intrarectal balloon segment is ensured even at such low filling pressures of, for example, 10 to 25 mbar.

A high pressure inside the rectum of a patient can lead to a distension of the patient's peritoneum, and this in turn can cause multiple potentially negative reactions of the patient's body, ranging from cardiac arrhythmia over increased blood pressure to sweating attacks, etc. In this regard, a reduction of the pressure inside of the intrarectal balloon segment to, for example, 30 to 50 mbar, reduces undesired harmful secondary effects on the patient's body.

Furthermore, the patient is protected against painful sensations due to any overexpansion of the rectum.

This pneumatic expansion stimulus that can be well controlled by the user can also reduce the necessary amount of infusion fluid in many cases, which in turn can considerably shorten the time needed to evacuate the infusion from the rectum, thereby ultimately opening up the method to many users as a therapeutic option for self-administration.

A non-return element that prevents any retrograde backflow of fluid directed away from the patient through the catheter may be inserted in a duct of the catheter shaft, or integrated into a tubing line, which connects the catheter shaft with a fluid containing containment.

Preferably, the non-return element in the duct is a thin-walled tube element having a wall thickness of 5 to 15 micrometers, and having the diameter of the duct, wherein the distal end of the tube lies freely in the duct over a length of approximately 5 to 10 mm and its proximal end is sealingly connected to the inner wall of the duct, thereby ensuring that the tube element opens in the presence of a flow of medium through the duct toward the tip and the tube element closes by collapsing in the presence of flow in the opposite direction.

The optional combined pneumatic/liquid triggering of reflexes described here eliminates the need for elaborate technical implementation of irrigation appliances, since in the preferred use case the necessary irrigation volume can be kept so small that it can be connected directly, as a compact, ready-to-use solution, via a fixed feed conduit to an inflow catheter designed according to the invention, and thus, in an ideal manner, be used as a disposable product.

It is recommended that the catheter is an inflow catheter and is connected by a fixed tube connection to a container for the irrigation fluid as a ready-to-use, disposal product.

It is an option of this invention to reduce the applied volume of an irrigation fluid to only a few milliliters, which is known as a micro-enema. Micro-enema solutions are typically measuring 5 to 15 ml of volume and are containing at least a chemically irritating phosphoric salt, preparing the mucosal response to a triggering stimulus. Such small volumes can be combined with the catheter and a catheter-tip lubricating gel in a compact sized, application ready, tear open package.

The invented catheter shall also be connectable to standard, commercially available mini-enemas, typically measuring 80 to 120 ml of volume. The catheter integrated tube component, connecting the catheter to a fluid reservoir, shall have a funnel shaped connecting element, allowing for multi-type connection of such enema units. The connecting element shall also enable a connection to standard, gravity driven irrigation bags, that are typically used for high-volume irrigation, exceeding 200 ml of fluid.

It is within the scope of the invented device, that it can be optionally applied without use of any irrigation fluid, triggering a coordinated colo-rectal defecation reflex solely by a single step or a multiple step pneumatic dilation of the rectum and/or the anal sphincter.

The invention further recommends that a diameter of an irrigation canal of the device is reduced in its dimension to a volume of equal or less than 1 ml, or to a volume of less than 0.5 ml, so as to minimize the volume loss inside this volume.

The fixed tube connection may be equipped with a breakable seal and/or with a non-return valve.

The device according to the invention may further comprise a funnel-like connector element at the proximal end of a tube connection, allowing for connection with an insertion tubing/element of ready-to-use, off-shelf enema solutions, as well as for connecting a standard irrigation container/system to the device, by a conical connector.

The inflow catheter according to the invention further comprises a preferably fixedly glued-in filling conduit for charging the catheter balloon with filling pressure. The unit for filling the balloon is preferably implemented in reusable form and can be configured as a hand-operated pump balloon connectable via a coupling or alternatively a pumped balloon with a pressure-indicating manometer. Alternatively, volume-controlled filling of the balloon is also conceivable. Regardless of the filling arrangement, the user can determine by stepwise approximation the degree of filling of the balloon that is optimal for him, that which is best adapted to his individual anatomy and his current reflex status.

Preferably, the device according to the invention comprises a disposable catheter, which is connectable via a filling conduit to a filling device, or to a filling device which is implemented as a manually operable pump balloon with a manometer indicating a balloon filling pressure.

Furthermore, the device according to the invention may comprise a manometer indicating a balloon filling pressure, wherein the manometer preferably comprises a scale on which filling pressure ranges required for the use of the catheter are specified by suitable markings.

The invention may be further characterized by a valve element that limits a balloon pressure and is disposed in or on a reusable filling device or is integrated into a balloon filling conduit of the catheter or into a catheter-bag unit.

A preferred embodiment of the invention may comprise a user-adjustable decompression mechanism, that when being released, opens the balloon in a pressurized state permanently to an ambient surrounding, once a certain pressure inside the balloon has been reached or exceeded, whereby a pressure increase inside the balloon is configured to be caused by a peristaltic contraction of the colo-rectal bowel segment, due to the released coordinated defecation reflex.

To ensure the most practicable catheter insertion possible and the reliable transanal placement of the tapered portion of the dumbbell-shaped balloon element, the catheter shaft is preferably equipped in the proximal, preanal region with gripping depressions to accommodate the fingers gripping the catheter during insertion. If the user has no sensitivity in the pelvic or anal region, he guides the catheter in with his fingers until they abut the anus, and can thus avoid uncontrolledly deep insertion.

Gripping depressions may be disposed on the catheter shaft in direct proximal adjacency to a rear balloon fixation line.

An envelope of the evacuated, ready-to-use catheter balloon can be placed on the shaft or clings closely thereto in such fashion that the intrarectal and the transanal balloon sections lie in a shaft region between the respective fixations of the upper and lower balloon ends, and a preanal balloon portion extends in the proximal direction in a pocket-like manner over gripping depressions.

Furthermore, the catheter shaft can have a waist in a region of its transanal placement inside the anal canal.

An embodiment of the invention may be configured such that, in an inflated, but unpressurized state of the balloon, the difference between the first radius of the intrarectal balloon segment and the third radius of the tapered transanal balloon section is equal to a second difference, and further wherein the second difference is greater than the distance between the distal front side of the catheter and the distal fixation point of the balloon to the catheter shaft; wherein the catheter balloon or at least the intrarectal balloon segment is pre-formed into a working dimension associated with a filling state characterized by an initial fill volume or an initial filling pressure, in which the filling pressure required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex by generating a reflex-triggering stimulus of an intensity which can, determined by the user, substantially exceed the intensity achievable by an irrigation with fluid media; wherein the catheter balloon or the intrarectal balloon segment has a compliance reserve; wherein the catheter balloon or the intrarectal balloon segment of the catheter balloon distends elastically from the pre-formed working dimension up to a final dimension; and wherein further distention of the catheter balloon or the intrarectal balloon segment of the catheter balloon is not possible once the catheter balloon or intrarectal balloon segment has distended to its final dimension, and no further dimensional expansion is allowed, in order to avoid uncontrolled dilation and trauma.

An embodiment of the invention may be configured such that the catheter balloon or at least the intrarectal balloon segment is pre-formed beyond a working dimension associated with a filling state characterized by an initial fill volume or an initial filling pressure, in which the filling pressure in the catheter balloon required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex by generating a reflex-triggering stimulus of an intensity which can, determined by the user, substantially exceed the intensity achievable by an irrigation with fluid media; and wherein the catheter balloon or the intrarectal balloon segment is made of a non-volume-expandable, non-elastic material, such that no further dimensional expansion is allowed beyond a final dimension to which the catheter balloon or the intrarectal balloon segment is pre-formed, in order to avoid uncontrolled dilation and trauma.

An embodiment of the invention may be configured such that the catheter balloon or at least the intrarectal balloon segment is pre-formed into a working dimension associated with a filling state characterized by an initial fill volume or an initial filling pressure, in which the filling pressure required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex; wherein the catheter balloon or the intrarectal balloon segment has a compliance reserve; wherein the catheter balloon or the intrarectal balloon segment of the catheter balloon distends elastically; wherein the transanal balloon segment expands by less than 20% at a pressure increase from 30 to 100 mbar, and wherein gripping depressions (12) are disposed on the catheter shaft in direct proximal adjacency to a rear balloon fixation line (14).

An embodiment of the invention may be configured such that the catheter balloon or at least the intrarectal balloon segment is pre-formed into a working dimension associated to with filling state characterized by an initial fill volume or an initial filling pressure, in which the filling pressure required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex;

wherein the catheter balloon or the intrarectal balloon segment has a compliance reserve; wherein the catheter balloon or the intrarectal balloon segment of the catheter balloon distends elastically from the pre-formed working dimension up to a final dimension and is designed to expand by more than 15%, at a pressure increase from 30 to 100 mbar up to a final dimension, where distention comes to a rest and no further dimensional expansion is allowed, in order to avoid uncontrolled dilation and trauma; and wherein the catheter balloon is connectable via a filling conduit (34) to a filling device (27) which is implemented as a manually operable pump balloon with a manometer indicating a balloon filling pressure.

An embodiment of the invention may be configured such that the catheter balloon or at least the intrarectal balloon segment is pre-formed into a working dimension associated to with filling state characterized by an initial fill volume or an initial filling pressure, in which the filling pressure required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex; wherein the catheter balloon or the intrarectal balloon segment has a compliance reserve; wherein the catheter balloon or the intrarectal balloon segment of the catheter balloon distends elastically from the pre-formed working dimension up to a final dimension and is designed to expand by more than 15%, at a pressure increase from 30 to 100 mbar up to a final dimension, where distention comes to a rest and no further dimensional expansion is allowed, in order to avoid uncontrolled dilation and trauma; wherein the catheter balloon is connectable via a filling conduit (34) to a filling device (27) which is implemented as a manually operable pump balloon with a manometer indicating a balloon filling pressure; and further wherein a user-adjustable decompression mechanism (37), that when being released, opens the balloon in a pressurized state permanently to an ambient surrounding, once a certain pressure inside the balloon has been reached or exceeded, or a valve element that limits a balloon pressure, is disposed in or on a filling device (27) or is integrated into a balloon filling conduit (34) of the catheter.

A method according to the invention for intermittently triggering a coordinated defecation reflex in the rectum or colon of a patient comprises the following steps:
providing a catheter comprising an inflexible catheter shaft and a preformed inflatable balloon, the inflatable balloon comprising at least an intrarectal balloon segment having, in an inflated, but unpressurized state of the balloon, a generally spherical or discoid shape and a maximal first radius, and which is adapted to be placed intrarectally; wherein the inflatable balloon is fixed onto the catheter shaft only at fixation points located at the ends of the inflatable balloon, wherein both ends of the inflatable balloon taper to a shaft dimension of the catheter shaft supporting the inflatable balloon, with the distal end of the balloon having a tapered second radius; and wherein, in an inflated, but unpressurized state of the balloon, the difference between the first radius of the intrarectal balloon segment and the second radius of the distal tapered balloon end is equal to a first difference, and further wherein the first difference is greater than a distance between a distal front side of the catheter and a distal fixation point, where the tapered distal end of the balloon is in contact with to the catheter shaft and is fixed thereto, wherein the distal front side of the catheter is at a distalmost extension of the catheter shaft or of a distal tip piece of the catheter shaft;
inserting the catheter in the rectum or colon of a patient;
pressurizing the balloon to a pressure exceeding 30 mbar, whereby at least a portion of the distal intrarectal balloon segment engages and expands a side wall of the rectum or colon of the patient, whereby to trigger a coordinated defecation reflex in the rectum or colon of the patient.

The aforementioned method can be further improved in that the catheter balloon comprises, additionally to the intrarectal balloon segment, a tapered transanal balloon section disposed proximally adjacent to the intrarectal balloon segment, the tapered transanal balloon section having a second radius, and wherein the first radius is larger than the second radius.

The aforementioned method can be further improved in that (i) in the pressurized state of the inflatable balloon at 30 mbar, the difference between the first radius of the intrarectal balloon segment and a third radius of the transanal balloon section is equal to a second difference, and (ii) in the pressurized state of the inflatable balloon at 100 mbar, the difference between the first radius of the intrarectal balloon segment and a third radius of the transanal balloon section is equal to a third difference, and further wherein the second difference is less than the third difference.

Preferably, in case of an increased pressure in the balloon beyond 100 mbar, the difference between the larger radius of the distal terminal balloon section and the reduced radius of the proximally adjacent, tapered balloon section will approximate a constant value.

The aforementioned method can be further improved in that the tapered transanal balloon section being adapted to be placed transanally is pre-formed to a diameter of 2 to 3.5 cm.

In the above-mentioned method, the tapered transanal balloon section being adapted to be placed transanally expands preferably by less than 20%, or by less than 15%, at a pressure increase from 30 to 100 mbar.

In the above-mentioned method, the intrarectal balloon segment being adapted to be placed intrarectally expands preferably by more than 15%, or by more than 20%, at a pressure increase from 30 to 100 mbar.

Within the method according to the invention, it is recommended to use a balloon which is made from polyurethane material, or from a material with the same compliance and elasticity characteristics as polyurethane.

The aforementioned method can be further improved in that a material of the balloon has a Shore durometer of 60A to 75A, or of 80A to 95A, or of 55D to 60D.

The aforementioned method can be further improved in that a wall thickness in the intrarectal balloon segment adapted to be placed intrarectally is 3 to 6 times thinner than a wall thickness in the tapered transanal balloon section adapted to be placed transanally, or is 4 to 5 times thinner than the wall thickness in the tapered transanal balloon section.

For the method according to the invention, it is recommended that a wall thickness in the intrarectal balloon segment adapted to be placed intrarectally is 10 to 30 μm, or is 15 to 25 μm.

For the method according to the invention, it is recommended that a wall thickness in the tapered transanal balloon section adapted to be placed transanally is 30 to 180 μm, or is 60 to 125 μm.

In the method according to the invention, the tapered transanal balloon section may be configured to terminate flush with the anus.

For the method according to the invention, it is recommended that the length of the tapered transanal balloon section is designed such that in case of a placement of the device in a patient, the tapered transanal balloon section shall project over the anus to form a pre-anal segment, wherein the pre-anal segment of the balloon is pre-formed into a conical balloon section having a conical shape with a distal end and a proximal end, wherein a pre-formed diameter at the proximal end of the conical balloon section is 30 to 70% larger, or 40 to 60% larger, than a pre-formed diameter at the distal end of the conical balloon section.

For the method according to the invention, it is recommended that the intrarectal balloon segment adapted to be placed intrarectally and the tapered transanal balloon section adapted to be placed transanally are manufactured separately, or are manufactured from different materials and/or with different characteristics, or are manufactured from materials with different material characteristics like different durometers.

For the method according to the invention, the tapered transanal balloon section adapted to be placed transanally can have less volume-expandable characteristics and/or less elasticity than the intrarectal balloon segment adapted to be placed intrarectally.

The aforementioned method can be further improved in that the intrarectal balloon segment adapted to be placed intrarectally is made from polyurethane with a Shore hardness of 80A to 90A.

Additionally, the aforementioned method can be further improved in that the tapered transanal balloon section adapted to be placed transanally is made from polyurethane with a Shore hardness of 95A or of 55D to 60D.

Within the method according to the invention, the inflatable balloon may have a waisted shape, comprising the intrarectal balloon segment of the first radius and of generally spherical or discoid shape, a proximal terminal balloon section of a fourth radius and of generally spherical or discoid shape, and, disposed between them, the tapered transanal balloon section having a third radius, with the tapered balloon section of the device being adapted to be placed transanally, and the intrarectal balloon segment being adapted to be placed intrarectally, and the proximal terminal balloon section being adapted to be placed extracorporeally, and wherein the fourth radius is larger than the third radius.

For the method according to the invention, it is recommended that the ends of the inflatable balloon, beginning at an inflection point of their longitudinal section, are inwardly invaginated, or inverted, and in the inverted or invaginated state are fixed on an outer jacket surface of the catheter shaft.

The above-mentioned method can be further improved in that the ends of the inflatable balloon are fixed on an outer jacket surface of the catheter shaft such that, at a transanally placement of the balloon in situ, the balloon waist seals inside and against the anal canal in a radial direction on all sides.

The above-mentioned method can be further improved in that, at a transanally placement of the balloon in situ, the catheter shaft can be displaced inside the anal canal by a tug or push on a tube connection at the catheter shaft by which the catheter is guided into the anal canal.

As the balloon is filled during the method according to the invention, a distal tip of the catheter shaft, in a non-displaced resting state, can retract into a protected, injury-preventing position inside the distal terminal balloon section adapted to be placed intrarectally and, in so doing comes to lie entirely proximally of a distal apex of a circle about a center point which is located on an axis of symmetry and whose tangent in an inflection point corresponds to a tangent to a non-invaginated longitudinal section of the balloon.

The above-mentioned method can be further improved in that the distal tip of the catheter shaft in its non-deflected, resting state comes to lie entirely proximally of a plane Z which is intersected perpendicularly by an axis X of symmetry and which is fully tangent distally to the intrarectal balloon section in an inflated, but unpressurized state of the balloon.

The above-mentioned method can be further improved in that the tapered transanal balloon section is separated from each of the approximately spherical or discoid balloon sections by a respective inflection point of a curvature of the balloon along a longitudinal direction and in a non-expanded filled state has an axial length A that is larger than its minimum diameter.

The method according to the invention is further characterized by invaginated or inverted balloon ends which, in the non-expanded filled state, each have an axial extent B₁, B₂ that is greater than their minimum diameter.

The above-mentioned method can be further improved in that in the non-expanded filled state, a sum of the axial extents B₁, B₂ of respective inversions of the two balloon ends are at least equal to the length of the tapered balloon section A:

B₁+B₂≥A.

If a catheter tip projects by a dimension C past a forward fixation line of a forward balloon end on the catheter shaft, the method according to the invention may further be characterized in that an inversion depth B is greater than or equal to half of length A plus the length C of the projecting tip piece:

B₁=B₂≥A/2+C,

or alternatively:

B₁=B₂≥A/2+C/2.

Upon performing the method according to the invention, even in case of an axial deflection of the catheter shaft toward the bowel in response to forces that typically occur during use, the catheter tip does not move more than a distance W beyond an apex of the forward balloon radius, and distance W is obtained from a distance between a first apex and a second apex, wherein the second apex is defined as the forward intersection point of the axis X of symmetry with a circle of diameter D around a center point on the axis X of symmetry at a level of the attachment of the intrarectal balloon section to the catheter shaft.

The above-mentioned method can be further improved in that an envelope of the evacuated, ready-to-use catheter balloon is placed on the shaft or clings closely thereto in such fashion that the intrarectal and the transanal balloon sections lie in a shaft region between the respective fixations of the upper and lower balloon ends, and a preanal balloon portion extends in the proximal direction in a pocket-like manner over gripping depressions.

The above-mentioned method can be further improved in that gripping depressions are disposed on the shaft in direct proximal adjacency to a rear balloon fixation line.

Another feature of the method according to the invention is that the catheter shaft has a waist in a region of its transanal placement inside the anal canal.

The above-mentioned method can be further improved by use of a non-return element that prevents any retrograde backflow of fluid directed away from the patient through the catheter, which non-return element can be inserted in a duct of the catheter shaft, or integrated into a tubing line, which connects the catheter shaft with a fluid containing containment.

The method according to the invention receives a further improvement in that the non-return element in the duct is a thin-walled tube element having a wall thickness of 5 to 15 micrometers, and having the diameter of the duct, wherein the distal end of the tube lies freely in the duct over a length of approximately 5 to 10 mm and its proximal end is sealingly connected to the inner wall of the duct, thereby ensuring that the tube element opens in the presence of a flow of medium through the duct toward the tip and the tube element closes by collapsing in the presence of flow in the opposite direction.

As recommended for the method according to the invention, the catheter can be an inflow catheter and may be connected by a fixed tube connection to a container for the irrigation fluid as a ready-to-use, disposal product.

Small volumes of fluid as e.g. 5 to 15 ml can be squeezed out from the container into the rectum of a patient, as a micro-enema.

In the scope of the method according to the invention, a diameter of an irrigation canal of the device can be reduced in its dimension to a volume of equal or less than 1 ml, or to a volume of less than 0.5 ml, so as to minimize the volume loss inside this volume.

The fixed tube connection may be equipped with a breakable seal and/or with a non-return valve.

Within the scope of the method according to the invention, a funnel-like connector element may be provided at the proximal end of a tube connection, allowing for connection with an insertion tubing/element of ready-to-use, off-shelf enema solutions, as well as for connecting a standard irrigation container/system to the device, by a conical connector.

In case the method according to the invention uses a disposable catheter, such disposable catheter may be connectable via a filling conduit to a filling device, or to a filling device which is implemented as a manually operable pump balloon with a manometer indicating a balloon filling pressure.

In case the method according to the invention uses a manometer indicating a balloon filling pressure, such manometer may comprise a scale on which filling pressure ranges required for the use of the catheter are specified by suitable markings.

The above-mentioned method can be further improved by a valve element that limits a balloon pressure and is disposed in or on a reusable filling device or is integrated into a balloon filling conduit of the catheter or into a catheter-bag unit.

In the scope of the method according to the invention, it is further recommended to provide a user-adjustable decompression mechanism, that when being released, opens the balloon in a pressurized state permanently to an ambient surrounding, once a certain pressure inside the balloon has been reached or exceeded, whereby a pressure increase inside the balloon is configured to be caused by a peristaltic contraction of the colo-rectal bowel segment, due to the released coordinated defecation reflex.

The above-mentioned method can be further improved in that, in an inflated, but unpressurized state of the balloon, the difference between the first radius of the intrarectal balloon segment and the third radius of the tapered transanal balloon section is equal to a second difference, and further wherein the second difference is greater than a the distance between a the distal front side of the catheter and a the distal fixation point of the balloon to the catheter shaft; wherein the catheter balloon or at least the intrarectal balloon segment is pre-formed into a working dimension associated with a filling state, characterized by an initial fill volume or an initial filling pressure, in which the filling pressure required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex by generating a reflex-triggering stimulus of an intensity which can, determined by the user, substantially exceed the intensity achievable by an irrigation with fluid media; wherein the catheter balloon or the intrarectal balloon segment has a compliance reserve; wherein the catheter balloon or the intrarectal balloon segment of the catheter balloon distends elastically from the pre-formed working dimension up to a final dimension; and wherein further distention of the catheter balloon or the intrarectal balloon segment of the catheter balloon is not possible once the catheter balloon or intrarectal balloon segment has distended to its final dimension, and no further dimensional expansion is allowed, in order to avoid uncontrolled dilation and trauma.

It is further recommended that the catheter balloon or at least the intrarectal balloon segment is pre-formed beyond a working dimension associated with a filling state characterized by an initial fill volume or an initial filling pressure, in which the filling pressure in the catheter balloon required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex by generating a reflex-triggering stimulus of an intensity which can, determined by the user, substantially exceed the intensity achievable by an irrigation with fluid media; and wherein the catheter balloon or the intrarectal balloon segment is made of a non-volume-expandable, non-elastic material, such that no further dimensional expansion is allowed beyond a final dimension to which the catheter balloon or the intrarectal balloon segment is pre-formed, in order to avoid uncontrolled dilation and trauma.

The above-mentioned method can be further improved in that the catheter balloon or at least the intrarectal balloon segment is pre-formed into a working dimension associated with a filling state, characterized by an initial fill volume or an initial filling pressure, in which the filling pressure required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex; wherein the catheter balloon or the intrarectal balloon segment has a compliance reserve; wherein the catheter balloon or the intrarectal balloon segment of the catheter balloon distends elastically; wherein the transanal balloon segment expands by less than 20%, at a pressure increase from 30 to 100 mbar, and wherein gripping depressions are disposed on the catheter shaft in direct proximal adjacency to a rear balloon fixation line.

Furthermore, a preferred method according to the invention is characterized in that the catheter balloon or at least the intrarectal balloon segment is pre-formed into a working dimension associated with a filling state, characterized by an initial fill volume or an initial filling pressure, in which the filling pressure required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex; wherein the catheter balloon or the intrarectal balloon segment has a compliance reserve; wherein the catheter balloon or the intrarectal balloon segment of the catheter balloon distends elastically from the pre-formed working dimension up to a final dimension and is designed to expand by more than 15%, at a pressure increase from 30 to 100 mbar up to a final dimension, where distention comes to a rest and no further dimensional expansion is allowed, in order to avoid uncontrolled dilation and trauma, and wherein the catheter balloon is connectable via a filling conduit to a filling device which is implemented as a manually operable pump balloon with a manometer indicating a balloon filling pressure.

Finally, it is within the scope of the method according to the invention that the catheter balloon or at least the intrarectal balloon segment is pre-formed into a working dimension associated with a filling state, characterized by an initial fill volume or an initial filling pressure, in which the filling pressure required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex; wherein the catheter balloon or the intrarectal balloon segment has a compliance reserve; wherein the catheter balloon or the intrarectal balloon segment of the catheter balloon distends elastically from the pre-formed working dimension up to a final dimension and is designed to expand by more than 15%, at a pressure increase from 30 to 100 mbar up to a final dimension, where distention comes to a rest and no further dimensional expansion is allowed, in order to avoid uncontrolled dilation and trauma, wherein the catheter balloon is connectable via a filling conduit to a filling device which is implemented as a manually operable pump balloon with a manometer indicating a balloon filling pressure, and further wherein a user-adjustable decompression mechanism, that when being released, opens the balloon in a pressurized state permanently to an ambient surrounding, once a certain pressure inside the balloon has been reached or exceeded, or a valve element that limits a balloon pressure, is disposed in or on a filling device or is integrated into a balloon filling conduit of the catheter.

### BRIEF DESCRIPTION OF THE DRAWINGS

Further features, characteristics, advantages and effects of the invention will become apparent from the following description of preferred embodiments of the invention and by reference to the drawing. Therein:

FIG. 1a shows an embodiment of a catheter according to the invention in a schematic longitudinal section, the balloon ends being mounted on the catheter shaft in an inverted manner according to the invention;

FIG. 1b shows the geometric structure of the apex of the distal balloon radius;

FIG. 1c shows the geometric structure of the distal plane tangent to the intrarectal balloon section;

FIG. 2 is a representation similar to FIG. 1a of another embodiment of the invention, in which a catheter tip extends beyond the forward fixation line of the end of the balloon shaft;

FIG. 3 is a representation similar to FIG. 2 showing additional auxiliary lines, such that the reference point for determining the inversion depth B is not the forward balloon radius, but rather the largest diameter D of the intrarectal balloon segment;

FIG. 4 shows a shaped balloon envelope in the unfilled state, provided in a particularly advantageous manner for rectal insertion and reliable transanal placement and unfolding of the balloon;

FIG. 5 shows a modified embodiment of the invention with a catheter shaft waisted in its transanal section;

FIG. 6 shows a manually operable pump manometer with a pressure scale equipped for multi-stage, sequential filling of the catheter balloon;

FIG. 7 shows a further modified embodiment of the invention with an infusion container fixedly connected to the catheter shaft and a filling tube for charging the catheter balloon with filling pressure;

FIG. 7a shows another modified embodiment of the invention where an infusion container is releasably connected to the catheter shaft and can be replaced by another equipment; and

FIG. 8 shows a pressure decompressing module 37, that once the reflex is released and the bowel begins to contract, fully opens and decompresses the catheter balloon to environment.

### DETAILED DESCRIPTION OF THE PREFERRED EMBODIMENTS

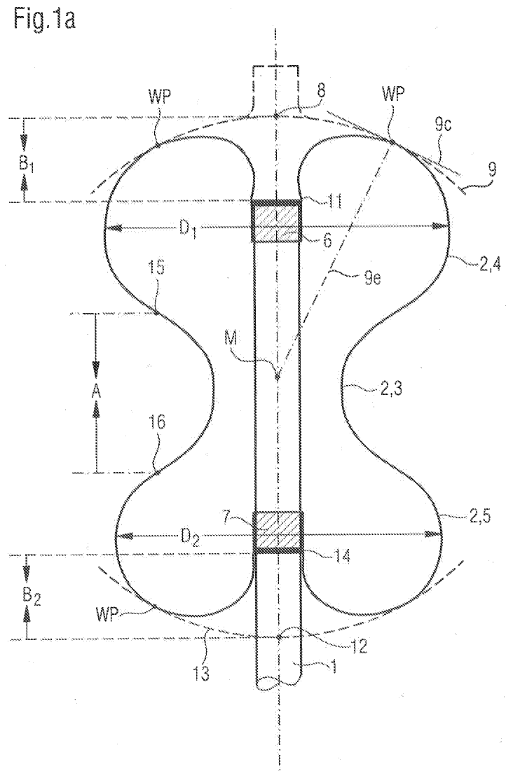

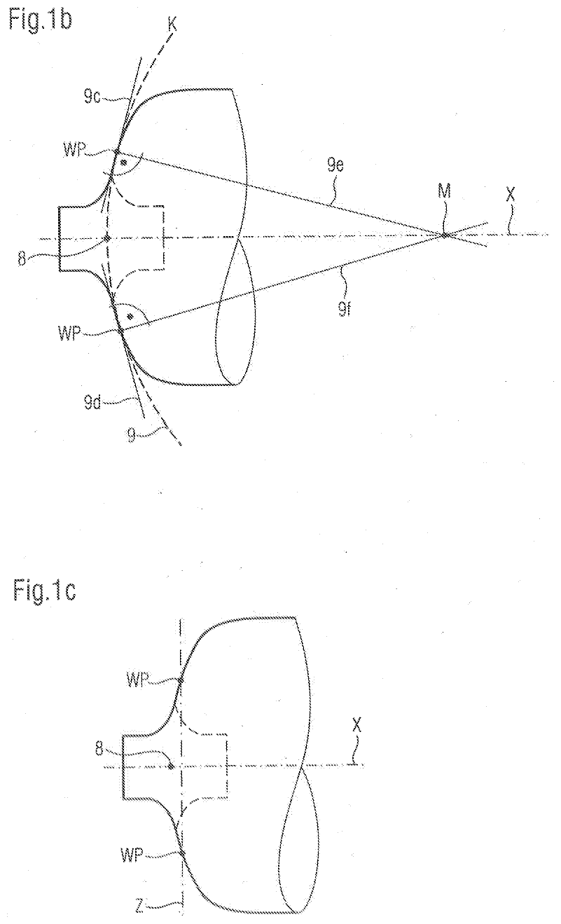

FIG. 1a shows a longitudinal section through an inflow catheter with a dumbbell- or hourglass-shaped balloon element 2 disposed at the distal end of the catheter shaft 1. The balloon element has, for example, a spherical or discoid expansion at each end. Disposed in the middle region between the terminal enlargements is a cylindrically or approximately cylindrically shaped segment 3 of reduced diameter, which continuously connects the intrarectal enlargement 4 to the preanal enlargement 5.

The balloon 2 terminally comprises two balloon shaft ends 6, 7 for attaching the balloon to the shaft. During the mounting of the balloon, the shaft ends 6 and 7 are invaginated (inverted) into the inside of the balloon by a defined magnitude B and in this position, in which they are displaced toward each other, are fixed on the shaft 1, for example by gluing or welding.

The sum of the magnitudes B of the inversions at both ends should be at least equal to the length of the tapered intermediate piece A (A smaller than/equal to the sum of the magnitudes B).

The inversion depth B at the end of the balloon facing the patient or the rectum corresponds to the distance from the apex 8 of the distal, intrarectal balloon radius 9 to the distal fixation line 11 of the balloon end 6 on the catheter shaft.

The distal radius 9 corresponds to the frontal radius during free, non-inverted unfolding of the completely filled but not pressurized balloon (broken line). An exemplary rule for geometrically determining the apex 8 to good approximation is illustrated in FIG. 1b. In particular, the drawing shows the two inflection points WP of the longitudinal section through the distal balloon end, which, by the inversion of the distal region of the balloon, result in the spherical or discoid or approximately semitoroidal region of the intrarectal section of the balloon. The normals 9e, 9f to the tangent to the longitudinal section of the balloon intersect with the longitudinal axis X of the catheter shaft at point M, and a circle K around this point M at the distance M-WP yields the forward apex 8, which marks the distal limit for the catheter shaft in its neutral, non-deflected initial position.

It can be seen in FIG. 1c that a straight line can also be passed through points WP; this straight line represents the plane Z which after invagination of the forward end of the balloon is now tangent distally to the intrarectal portion of the balloon and can also be taken as a measure of the distalmost position of the catheter shaft in its neutral, non-deflected initial position.

On the side of the balloon facing away from the patient, the inversion depth B corresponds to the distance from the apex 12 of the proximal, preanal balloon radius 13 to the proximal fixation line 14 of the balloon end 7 on the catheter shaft.

Radius 13 corresponds to the proximal radius when the balloon is freely unfolded without inversion. The geometric determination of apex 12 is similar to the approximation described in FIG. 1b.

The length of intermediate piece A is determined by determining the distance between the transitions of the shoulder radii 15 and 16 (inflection points) of the mutually facing shoulder surfaces of balloon segments 4 and 5.

The inversion depths, lengths and distances are each determined in the filled state under filling pressure, the filling pressure being so selected that the balloon unfolds completely but there is no elastic expansion of the balloon envelope.

The inversion depth B is calculated as follows: B>=A/2 (>=represents greater/equal).

In mounting the balloon on the catheter shaft, the respective points for the inversion of each of the balloon shaft ends are the apex 8 of the distal, intrarectal balloon radius 9 and the apex 12 of the proximal, preanal balloon radius 13.

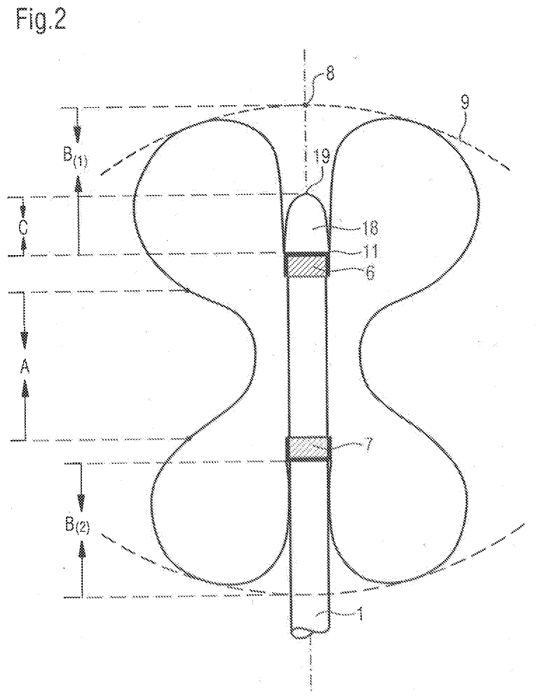

In the embodiment described in this figure, the distal fixation line 11 also corresponds to the distal end of the catheter shaft 1. The shaft terminates directly at the fixation line 11 and does not extend distally past this fixation line in the manner illustrated in FIG. 2.

FIG. 1b describes the geometric derivation of the apex of the distal balloon radius.

The forward balloon radius 9, frontally facing the intestinal lumen, of the intrarectal balloon segment 4 is illustrated as a broken line. It is constructed from the two inflection points 9a and 9b and the two inflection tangents 9c and 9d respectively belonging to these inflection points.

One of the two inflection points 9a or 9b is used to construct a straight line 9e or 9f that is normal to the respective inflection tangent 9c or 9d and intersects the corresponding inflection point 9a or 9b. The point of intersection of these straight lines 9e or 9f with the axis X of symmetry yields the center point of the circle K.

The circle K, and thus the forward balloon radius 9, results from the center point M of the circle and the inflection points 9a and 9b, which are on the circumference of the circle. In this derivation, apex 8 is obtained from the point of intersection of the circumference of the circle with the axis X of symmetry of the balloon.

For the description of the frontal apex 8 used below, the derivation of the farthest distally ranging point of the filled, unpressurized balloon envelope is performed, for purposes of simplification, using the point of intersection of the connecting line Z between the two inflection points 9a and 9b with the axis X of symmetry.

The catheter shaft 1 should be “inflexible”, that means, in case of an axial thrust force onto the catheter shaft upon its insertion into the anus, it should resist any kinking deformation. Furthermore, it should be radially stable, i.e. not allowing for a collapse of the catheter lumen. Additionally, the catheter shaft 1 should resist any twisting or torsion movement. On the other hand, in order to avoid lesions of the bowel wall, the catheter shaft 1 may have a minor ability of an axial bending deformation, but such axial bending ability should be limited, especially to a bending angle of not more than 45° over the entire length of the catheter shaft.

FIG. 2 shows, by way of example, how sections of the catheter shaft in the form of a tip piece 18 that extend beyond the distal fixation line 11 in a distal prolongation directed toward the bowel are to be considered in determining the inversion depth of the balloon ends 6 and 7, in order to ensure atraumatic inversion, according to the invention, of the distal end of the catheter in the intrarectal balloon during transanal placement of the filled catheter balloon. The length C of the tip piece 18 is defined as the distance from the forward fixation line 11 to the forward apex 19 of the tip piece.

Length B is increased in comparison to FIG. 1a by the magnitude of length C or C/2.

The corresponding inversion depth B is preferably calculated taking into account a tip piece in the form: B>=A/2+C.

Alternatively hereto, the corresponding inversion depth B can, less preferably, be obtained considering a tip piece according to the relation: B>=A/2+C/2.

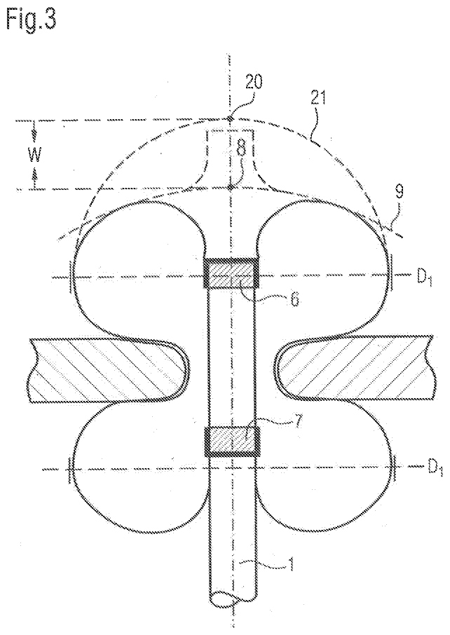

FIG. 3 illustrates another, alternative rule for establishing the inversion depth B of the balloon shaft ends 6 and 7 on the catheter shaft 1 in relation to the distal catheter shaft end 11, 19.

This rule particularly considers a possible axially oriented deflection of the catheter shaft inside the filled, transanally placed balloon. In the context of the inventively described inversion of the balloon shaft ends on the catheter shaft supporting the balloon, such deflection of the shaft in the longitudinal axis could cause the distal end of the catheter shaft to be deflected toward the bowel, thus creating a potential risk of perforation.

The maximum distal deflection W of the forward fixation line 11 is defined as a distance that emanates from the apex 8 of the radius 9, forming a distally directed prolongation of the longitudinal axis of the shaft, and extends to the apex 20 of a radius 21, said radius 21 being constructed over the largest diameter D of the intrarectal balloon segment 4.

If the catheter shaft has a tip piece 18 that extends beyond line 11, the maximum deflection path W should be correspondingly selected so that upon maximum deflection W of the shaft, the tip 19 of the tip piece does not extend past radius 21.

The radius 21 defined by the largest diameter D in the intrarectal balloon segment constitutes a fundamentally relevant boundary line for distal portions of the catheter shaft. In the event of lateral tilting of the shaft longitudinal axis of the transanally placed catheter shaft, the fact that the maximum deflection W is referred to the largest balloon diameter D ensures that the catheter shaft tip (11, 19) still moves within the pivot radius 21 of balloon segment 4, thus preventing relatively well any potential traumatizing contact of the tip with the bowel wall adjacent the balloon.

In determining the inversion depth B, the particular ratio of the distance W to the radius 21 or of the diameter D on which it is based is preserved and the inversion depth B is adjusted accordingly as necessary.

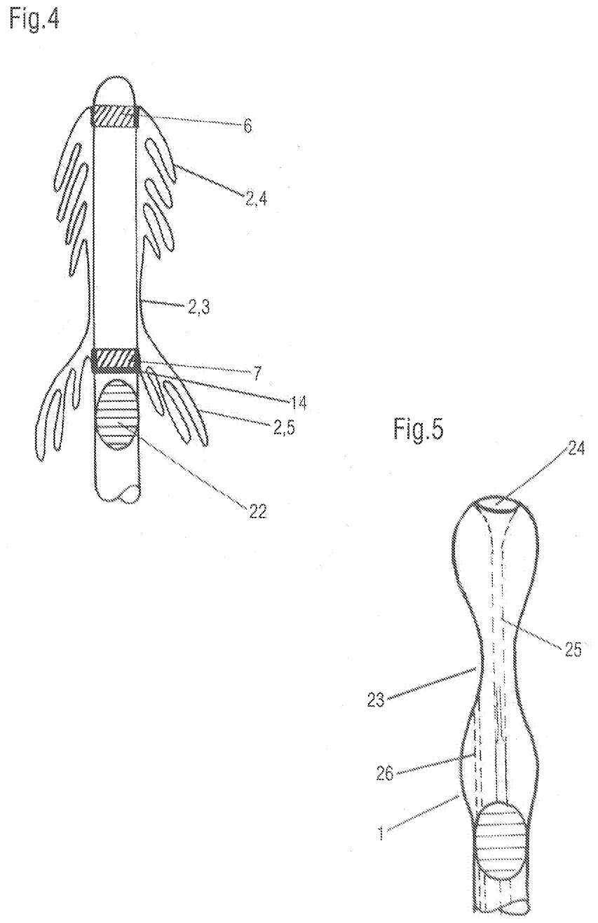

FIG. 4 shows the catheter balloon 2 in its emptied form, in which it lies against the catheter shaft ready for insertion in the anus. The envelope segments of the intrarectal balloon 4 and of the middle segment 3 cling to the shaft in folds.

In this state, the two envelope portions preferably come to lie approximately at the height of the line segment between the fixation points of the balloon ends 6 and 7 on the surface of the shaft. The envelope of the preanal balloon segment 5, by contrast, is preferably smoothed out in the proximal direction and protrudes past the fingers gripping the catheter for insertion, the preferred gripping point being located just proximal of the proximal fixation line 14. The gripping point 22 is preferably implemented as a depression-like receiving surface, one such preferably being provided on each of the opposite, 180°-apart shaft surfaces.

The evacuated balloon being fixed in such fashion, the user grips with his fingers under the envelope of segment 5, which envelope is smoothed out in a proximal direction, and guides the catheter into the rectum until the gripping fingers abut the external anus. A defined insertion depth is thereby ensured. It is further ensured that the intrarectal balloon section 4 is inserted into the rectal cavity, while the proximal balloon portion 5 comes to lie outside the anus (preanally). Reliable transanal positioning of the catheter therefore occurs as the waisted balloon is filled.

FIG. 5 shows a preferred implementation of the shaft body 1 that furnishes some degree of automatic positioning and securing of the shaft in the anus even when the catheter balloon has not yet been filled. The shaft 1 has for this purpose a likewise waisted shape, which is correspondingly tapered in the transanal region 23 and in effect locks the catheter shaft in a transanal position in this region after insertion. The catheter shaft preferably also has a distally terminal, funnel-like, atraumatically shaped opening 24 that connects to the duct 25 supplying the medium. In FIG. 5, the balloon 2, 3, 4, 5 is omitted to give an uncovered sight onto the shaft body 1.

In a particularly large-volume implementation of the intrarectal balloon segment or a longitudinal expansion of the balloon segment that reaches far into the rectum, the balloon segment, in the non-air-filled, ready-to-use state, can optionally be stuffed or packed partially into the opening 24. It then slips out of the opening as the inserted catheter is filled.

Given suitable implementation of the shaft material, the waist 23 can also predefine a certain kinkability of the shaft body, thus improving its atraumatic properties.

The filling of the balloon takes place through a separate duct 26 integral to the shaft.

To prevent backflow of irrigation fluid, the inflow catheter can is [sic] equipped with a non-return valve in the region of the fluid-conveying duct 25.

The valve can preferably consist of a thin-walled tube element having a wall thickness of few, preferably, 5 to 15 micrometers and the diameter of the duct 25, the distal end of the tube lying freely in the duct 25 over a length of approximately 5 to 10 mm and its proximal end being connected sealingly to the inner wall of the duct 25. As the medium flows through the duct in the direction of the tip, the tube element opens and allows the medium to flow freely. In response to flow in the reverse direction, the tube element collapses and closes up sealingly, preventing an effective backflow.

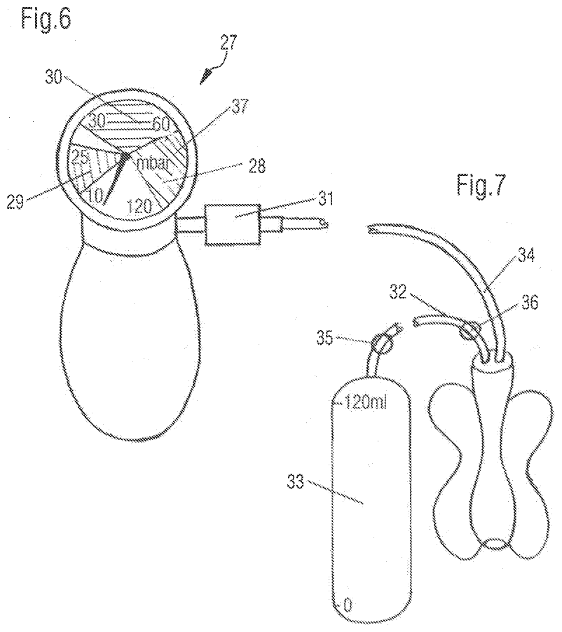

FIG. 6 schematically illustrates a pump manometer 27 equipped with a scale 28 suitable for the filling of the inventive catheter balloon in a single step or multiple steps or with a sequential increase in filling pressure. The scale preferably displays, on the one hand, an initial, low pressure range 29 (app. 10-25 mbar), which is set by the user after inserting the catheter and before introducing the infusion fluid, and which, given the inventive preshaping of the balloon envelope and its fixation on the shaft, is sufficient in most cases to ensure transanal anchoring and sealing of the catheter without a direct reflex-triggering effect on the bowel wall.

When the user then initiates an increase in the filling pressure in the balloon into range 30 (30-60 mbar, with increasing corresponding expansion of the rectal portions of the bowel wall) or into range 37 (60-120 mbar, with increasing additional expansion of the anal sphincter), the user is ultimately able to generate a trigger stimulus of largely reproducible intensity in order to trigger a defecation reflex. The user thus has the advantage of being able to avoid an evacuation reflex in the presence of an initially low balloon pressure merely having an anchoring and sealing action, and thus of being able to retain the infusion fluid in the bowel long enough for his individual needs, resulting in better dissolution or suspension of stool in the fluid. On the other hand, by intentionally causing an increase in pressure in the balloon, he can generate an intense, relatively prompt-acting reflex-triggering stimulus, which can, if necessary, exceed in intensity the triggering effect of a column of colorectal fluid.

The catheter balloon is preferably filled with air, through a fill line integrated into the shaft wall of the catheter body 1.

In addition to pressure-controlled filling of the balloon using a pump manometer, as illustrated in FIG. 6, volume-controlled filling can be achieved with the use of an additional syringe element that specifies the preferred fill volume by means of suitable markings on the syringe body. The filling of the balloon is preferably partial, in the form of one-step filling. In the ideal case, therefore, in loose, unexpanded form, the balloon lies against the structures of the bowel and the anus. The balloon envelope thus absorbs the forces acting on the balloon in the rectum, the anus and the preanal region and brings the balloon into the configuration in which it performs the anchoring and sealing function. The respective physiologically acting forces are absorbed by the catheter balloon and thus enable the balloon to be placed anorectally in as pressure-neutral a manner as possible, largely precluding undesired and premature triggering effects.

A volume-controlled filling of the balloon can also be a two-step process, in which incomplete filling is first performed and the balloon is then, in the second filling step, filled with a volume that has a triggering effect. The pressures developed in the transanal balloon in response to the particular volume should preferably fall within the pressure ranges (29, 30) described in FIG. 6, and are to be determined by and for the user on an individual basis, as in the case of pressure-controlled filling. The manometer can be equipped with a marking function 28a, allowing for marking the individually needed pressure values on the manometer scale.

To limit the filling pressure or prevent critically high balloon filling pressures, in the case of both manometer- and syringe-actuated filling a pressure limiting valve 31 can be interposed between the filling element and the catheter, to prevent, for example, balloon filling pressures over 120 mbar.

FIG. 7 shows a preferred embodiment of an inflow catheter, which transitions at the proximal end to two feed conduits that are preferably fixedly connected to the shaft, feed conduit 32 being fixedly connected to a preferably bag-like container 33 containing infusion medium, and thus constituting a ready-to-use unit comprised of catheter and medium.

The volume of the container 33 must be dimensioned in this case to accommodate approximately 80 to 120 ml of irrigation solution, which would correspond to a so called “mini-enema”. The relatively small quantity is squeezed out manually by the user and thus introduced into the rectum by repeated squeezing. To make it possible to grip the container even with impaired hand motor function, the container is preferably shaped as cylindrical, with a diameter of approximately 4-6 cm.

Optionally, the connected container takes only 5 to 15 ml of solution, corresponding to a so called “micro-enema”, typically being squeezed out between the thumb and the indicating finger. In order to prevent, that such small volumes are pooling inside the lumen of the irrigating canal, the diameter of the irrigating canal, including the tubing connection 32 and the irrigating canal inside the catheter shaft, is reduced in its dimensions to a volume of equal or less than 1 ml, or preferably less than 0.5 ml of volume, so that the micro-enema reaches the patients rectum without excessive loss inside the device.

Furthermore, as can be seen in FIG. 7a, connection can preferably be equipped with a seal 35 that can be broken by bending and that releases the irrigation solution. It is also advantageous to provide a non-return valve 36, which is integrated into the fluid-conveying arm of the ready-to-use device and which permits the directed emptying of the container without backflow. The connection optionally ends proximally with a funnel like connector 32a, allowing for a multi-fit connection to a variety of off-shelf, ready to use irrigation solutions.

Another feed conduit 34 can be connected directly to a filling syringe, a pump manometer or a pressure decompressing mechanism.

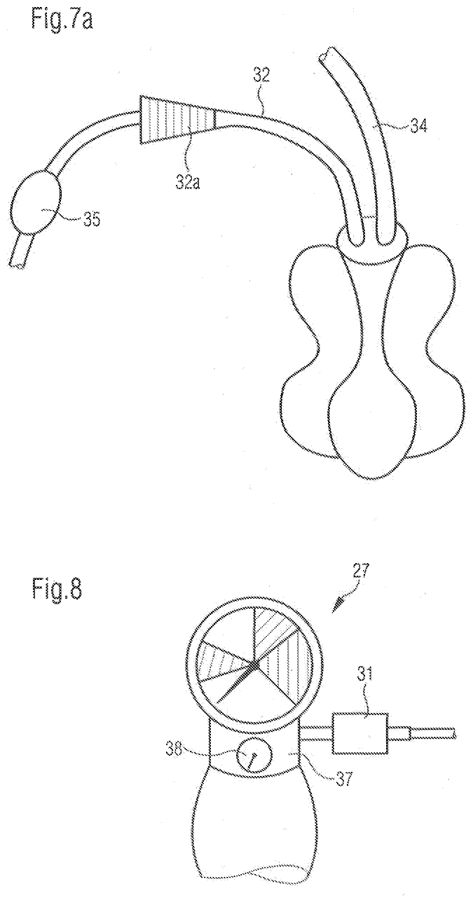

FIG. 8 shows a pressure decompressing module 37, that can be operated as a stand-alone device, or modularly integrated into a pump manometer as pictured in FIG. 6. The module works by e.g. a spring operated opening mechanism, which continuously opens the filling line 34 and the balloon to the ambient environment, fully decompressing the balloon. The threshold for opening the mechanism can be set by user individually to a certain balloon pressure, by an adjustable trigger knob 38. As pictured, the module can be positioned in between the scale and the pump balloon of the manometer. The pressure decompressing module is of particular benefit for e.g. spinal cord injury patients, not being able to sense stool urge or the onset of peristaltic bowel contraction. Once the reflex is released and the bowel begins to contract, the catheter “opens” and can be pulled out or expelled from the rectum.

The balloon is made from polyurethane material, or from a material with a similar compliance and elasticity characteristics. Preferably, when using polyurethanes, the balloon has a Shore A durometer of 80 to 95, preferably of 80 to 85.

The wall thickness in the enlarged distal terminal balloon section being adapted to be placed intrarectally is 3 to 6 times thinner than the wall thickness in the tapered balloon section being adapted to be placed transanally, especially 4 to 5 times thinner.

The wall thickness in the enlarged distal terminal balloon section being adapted to be placed intrarectally is 10 to 30 μm, especially 15 to 25 μm.

The thickness of the wall in the tapered balloon section being adapted to be placed transanally is 30 to 180 μm, especially 60 to 125 μm.

The use of only slightly volume-expandable (compliant) materials, such as, for example, polyurethane (PUR), for example of the specification Pellethane 2363 80A to 90A, Lubrizol Corp., is preferred, since these materials have good dimensional stability in the lowest wall thickness range, including as balloon films, in the pressure range of approximately 10 to 120 mbar.

Such thin-walled PUR balloon films formed into complex shape can preferably be produced by hot molding from previously extruded raw tubing material, which, with suitable stretching of the tube blank before tempering, permits a polymer orientation and lends the shaped balloon films exceptional mechanical strength.

It is also conceivable to use polyurethanes of low Shore hardness, for example in the range of 60 to 75A, to impart a volume-expandable behavior, with a relative loss of dimensional stability, to the catheter balloon in the wall thickness range of less than 40 μm, and preferably less than 15 μm, in the typical filling pressure range during use of 10 to 120 μm.

Alternatively, non-volume-expandable, non-elastic materials can be used, such as polyethylene, PVC or mixtures of the aforesaid materials with polyurethane.

Balloon films according to the invention can also be shaped directly from the extruded, still soft, largely amorphous tube molding compound (in-line molding), in which case the achievable strengths of the films are much lower than those of pre-extruded tubes and the attainable wall thicknesses are much higher than in the case of forming from pre-extruded material.

Dipping processes using liquid PVC or PUR materials can also be contemplated for production.

The welding together of single layers of film to form balloon bodies is also conceivable.

The connection of the balloon to the shaft body is effected by gluing, by thermal methods, or alternatively by shrinking the balloon ends onto the shaft body.

In the freely unfolded, unpressurized state, the balloon segments, in the unpressurized state, should have diameters and length dimensions of:
intrarectal: measuring a diameter of 40 to 80 mm, preferably 50 to 70 mm, measuring a length of 30 to 60 mm, preferably 35 to 45 mm;
transanal: measuring a diameter of 10 to 35 mm, preferably 20 to 25 mm, measuring a length of 20 to 50 mm, preferably 25 to 35 mm.

In addition to the transanal use of the inflow catheter, the embodiments according to the invention can also be used for placement of a transanally inserted drainage tube for, among other purposes, the ongoing drainage of stool from a patient's intestine. Further, the described catheter technique may also be contemplated for use in surgically created stomata/openings or other natural body openings.

1.-96. (canceled)

97. A device for intermittently triggering a coordinated defecation reflex in the rectum or colon of a patient, said device comprising a catheter with an inflexible catheter shaft and a preformed inflatable balloon, the inflatable balloon comprising at least an intrarectal balloon segment having, in an inflated, but unpressurized state of the balloon, a generally spherical or discoid shape and a maximal first radius, and being adapted to be placed intrarectally; wherein the inflatable balloon is fixed onto the catheter shaft only at fixation points located at the ends of the inflatable balloon, wherein both ends of the inflatable balloon taper to a shaft dimension of the catheter shaft supporting the inflatable balloon, with the distal end of the balloon having a tapered second radius; wherein, in an inflated, but unpressurized state of the balloon, the difference between the first radius of the intrarectal balloon segment and the second radius of the distal tapered balloon end is equal to a first difference, and further wherein the first difference is greater than a distance between a distal front side of the catheter and a distal fixation point, where the tapered distal end of the balloon is in contact with the catheter shaft and is fixed thereto; wherein the distal front side of the catheter is at a distalmost extension of the catheter shaft or of a distal tip piece of the catheter shaft; and wherein the inflatable balloon is configured such that when the inflatable balloon is in a pressurized state exceeding 30 mbar, at least a portion of the distal intrarectal balloon segment engages and expands a side wall of the rectum or colon of the patient, whereby to trigger a coordinated defecation reflex in the rectum or colon of the patient.

98. The device according to claim 97, wherein, the catheter balloon comprises, additionally to the intrarectal balloon segment, a tapered transanal balloon section disposed proximally adjacent to the intrarectal balloon segment, the tapered transanal balloon section having a third radius, and wherein the first radius is larger than the third radius.

99. The device according to claim 98, wherein (i) in the pressurized state of the inflatable balloon at 30 mbar, the difference between the first radius of the intrarectal balloon segment and a third radius of the transanal balloon section is equal to a second difference, and (ii) in the pressurized state of the inflatable balloon at 100 mbar, the difference between the first radius of the intrarectal balloon segment and the third radius of the transanal balloon section is equal to a third difference, and further wherein the second difference is less than the third difference.

100. The device according to claim 98, wherein, in case of an increased pressure in the balloon beyond 100 mbar, the difference between the larger radius of the distal terminal balloon section and the reduced radius of the proximally adjacent, tapered balloon section will approximate a constant value.

101. The device according to claim 98, wherein the tapered transanal balloon section being adapted to be placed transanally expands by less than 20%, or by less than 15%, at a pressure increase from 30 to 100 mbar.

102. The device according to claim 97, wherein the intrarectal balloon segment being adapted to be placed intrarectally expands by more than 15%, or by more than 20%, at a pressure increase from 30 to 100 mbar.

103. The device according to claim 97, wherein the balloon is made from polyurethane material, or from a material with the same compliance and elasticity characteristics as polyurethane.

104. The device according to claim 98, wherein the tapered transanal balloon section is configured to terminate flush with the anus.

105. The device according to claim 98, wherein the length of the tapered transanal balloon section is designed such that in case of a placement of the device in a patient, the tapered transanal balloon section shall project over the anus to form a pre-anal segment, wherein the pre-anal segment of the balloon is pre-formed into a conical balloon section having a conical shape with a distal end and a proximal end, wherein a pre-formed diameter at the proximal end of the conical balloon section is 30 to 70% larger, or 40 to 60% larger, than a pre-formed diameter at the distal end of the conical balloon section.

106. The device according to claim 98, wherein the intrarectal balloon segment adapted to be placed intrarectally and the tapered transanal balloon section adapted to be placed transanally are manufactured separately, or are manufactured from different materials and/or with different characteristics, or are manufactured from materials with different material characteristics like different durometers.

107. The device according to claim 98, wherein the inflatable balloon has a waisted shape, comprising the intrarectal balloon segment of the first radius and of generally spherical or discoid shape, a proximal terminal balloon section of a fourth radius and of generally spherical or discoid shape, and, disposed between them, the tapered transanal balloon section having a third radius, with the tapered balloon section of the device being adapted to be placed transanally, and the intrarectal balloon segment being adapted to be placed intrarectally, and the proximal terminal balloon section being adapted to be placed extracorporeally, and wherein the fourth radius is larger than the third radius.

108. The device according to claim 97, wherein the ends of the inflatable balloon, beginning at an inflection point of their longitudinal section, are inwardly invaginated, or inverted, and in the inverted or invaginated state are fixed on an outer jacket surface of the catheter shaft.

109. The device as in claim 98, characterized in that an envelope of the evacuated, ready-to-use catheter balloon is placed on the shaft or clings closely thereto in such fashion that the intrarectal and the transanal balloon sections lie in a shaft region between the respective fixations of the upper and lower balloon ends (6, 7), and a preanal balloon portion extends in the proximal direction in a pocket-like manner over gripping depressions (12).

110. The device as in claim 97, characterized in that gripping depressions (12) are disposed on the shaft in direct proximal adjacency to a rear balloon fixation line (14).

111. The device as in claim 97, characterized in that the catheter shaft has a waist (23) in a region of its transanal placement inside the anal canal.

112. The device as in claim 97, characterized in that inserted in a duct (25) of the catheter shaft, or integrated into a tubing line, which connects the catheter shaft with a fluid containing containment, is a non-return element that prevents any retrograde backflow of fluid directed away from the patient through the catheter.

113. The device as in claim 112, characterized in that the non-return element in the duct (25) is a thin-walled tube element having a wall thickness of 5 to 15 micrometers, and having the diameter of the duct (25), wherein the distal end of the tube lies freely in the duct (25) over a length of approximately 5 to 10 mm and its proximal end is sealingly connected to the inner wall of the duct (25), thereby ensuring that the tube element opens in the presence of a flow of medium through the duct toward the tip and the tube element closes by collapsing in the presence of flow in the opposite direction.

114. The device as in claim 97, characterized in that the catheter is an inflow catheter and is connected by a fixed tube connection (32) to a container (33) for the irrigation fluid as a ready-to-use, disposal product.

115. The device as in claim 114, whereby small volumes of fluid as e.g. 5 to 15 ml can be squeezed out from the container (33) into the rectum of a patient, as a micro-enema.

116. The device as in claim 114, whereby a diameter of an irrigation canal of the device is reduced in its dimension to a volume of equal or less than 1 ml, or to a volume of less than 0.5 ml, so as to minimize the volume loss inside this volume.

117. The device as in claim 97, comprising a disposable catheter, characterized in that the disposable catheter is connectable via a filling conduit (34) to a filling device (27), or to a filling device (27) which is implemented as a manually operable pump balloon with a manometer indicating a balloon filling pressure.

118. The device as in claim 97, comprising a manometer indicating a balloon filling pressure, characterized in that the manometer comprises a scale on which filling pressure ranges required for the use of the catheter are specified by suitable markings.

119. The device as in claim 97, characterized by a valve element that limits a balloon pressure and is disposed in or on a reusable filling device (27) or is integrated into a balloon filling conduit (34) of the catheter or into a catheter-bag unit.

120. The device as in claim 97, further comprising a user-adjustable decompression mechanism (37), that when being released, opens the balloon in a pressurized state permanently to an ambient surrounding, once a certain pressure inside the balloon has been reached or exceeded, whereby a pressure increase inside the balloon is configured to be caused by a peristaltic contraction of the colo-rectal bowel segment, due to the released coordinated defecation reflex.

121. The device according to claim 98, wherein, in an inflated, but unpressurized state of the balloon, the difference between the first radius of the intrarectal balloon segment and the third radius of the tapered transanal balloon section is equal to a second difference, and further wherein the second difference is greater than the distance between the distal front side of the catheter and the distal fixation point of the balloon to the catheter shaft; wherein the catheter balloon or at least the intrarectal balloon segment is pre-formed into a working dimension associated with a filling state characterized by an initial fill volume or an initial filling pressure, in which the filling pressure required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex by generating a reflex-triggering stimulus of an intensity which can, determined by the user, substantially exceed the intensity achievable by an irrigation with fluid media; wherein the catheter balloon or the intrarectal balloon segment has a compliance reserve; wherein the catheter balloon or the intrarectal balloon segment of the catheter balloon distends elastically from the pre-formed working dimension up to a final dimension; and wherein further distention of the catheter balloon or the intrarectal balloon segment of the catheter balloon is not possible once the catheter balloon or intrarectal balloon segment has distended to its final dimension, and no further dimensional expansion is allowed, in order to avoid uncontrolled dilation and trauma.

122. The device according to claim 97, wherein the catheter balloon or at least the intrarectal balloon segment is pre-formed beyond a working dimension associated with a filling state characterized by an initial fill volume or an initial filling pressure, in which the filling pressure in the catheter balloon required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex by generating a reflex-triggering stimulus of an intensity which can, determined by the user, substantially exceed the intensity achievable by an irrigation with fluid media; and wherein the catheter balloon or the intrarectal balloon segment is made of a non-volume-expandable, non-elastic material, such that no further dimensional expansion is allowed beyond a final dimension to which the catheter balloon or the intrarectal balloon segment is pre-formed, in order to avoid uncontrolled dilation and trauma.

123. The device according to claim 98, wherein the catheter balloon or at least the intrarectal balloon segment is pre-formed into a working dimension associated with a filling state characterized by an initial fill volume or an initial filling pressure, in which the filling pressure required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex; wherein the catheter balloon or the intrarectal balloon segment has a compliance reserve; wherein the catheter balloon or the intrarectal balloon segment of the catheter balloon distends elastically; wherein the transanal balloon segment expands by less than 20% at a pressure increase from 30 to 100 mbar, and wherein gripping depressions (12) are disposed on the catheter shaft in direct proximal adjacency to a rear balloon fixation line (14).

124. The device according to claim 97, wherein the catheter balloon or at least the intrarectal balloon segment is pre-formed into a working dimension associated to with filling state characterized by an initial fill volume or an initial filling pressure, in which the filling pressure required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex;
wherein the catheter balloon or the intrarectal balloon segment has a compliance reserve; wherein the catheter balloon or the intrarectal balloon segment of the catheter balloon distends elastically from the pre-formed working dimension up to a final dimension and is designed to expand by more than 15%, at a pressure increase from 30 to 100 mbar up to a final dimension, where distention comes to a rest and no further dimensional expansion is allowed, in order to avoid uncontrolled dilation and trauma;

125. A method for intermittently triggering a coordinated defecation reflex in the rectum or colon of a patient, said method comprising:
providing a catheter comprising an inflexible catheter shaft and a preformed inflatable balloon, the inflatable balloon comprising at least an intrarectal balloon segment having, in an inflated, but unpressurized state of the balloon, a generally spherical or discoid shape and a maximal first radius, and which is adapted to be placed intrarectally; wherein the inflatable balloon is fixed onto the catheter shaft only at fixation points located at the ends of the inflatable balloon, wherein both ends of the inflatable balloon taper to a shaft dimension of the catheter shaft supporting the inflatable balloon, with the distal end of the balloon having a tapered second radius; and wherein, in an inflated, but unpressurized state of the balloon, the difference between the first radius of the intrarectal balloon segment and the second radius of the distal tapered balloon end is equal to a first difference, and further wherein the first difference is greater than a distance between a distal front side of the catheter and a distal fixation point, where the tapered distal end of the balloon is in contact with to the catheter shaft and is fixed thereto, wherein the distal front side of the catheter is at a distalmost extension of the catheter shaft or of a distal tip piece of the catheter shaft;
inserting the catheter in the rectum or colon of a patient;
pressurizing the balloon to a pressure exceeding 30 mbar, whereby at least a portion of the distal intrarectal balloon segment engages and expands a side wall of the rectum or colon of the patient, whereby to trigger a coordinated defecation reflex in the rectum or colon of the patient.

126. The method according to claim 125, wherein the catheter balloon comprises, additionally to the intrarectal balloon segment, a tapered transanal balloon section disposed proximally adjacent to the intrarectal balloon segment, the tapered transanal balloon section having a second radius, and wherein the first radius is larger than the second radius.

127. The method according to claim 126, wherein (i) in the pressurized state of the inflatable balloon at 30 mbar, the difference between the first radius of the intrarectal balloon segment and a third radius of the transanal balloon section is equal to a second difference, and (ii) in the pressurized state of the inflatable balloon at 100 mbar, the difference between the first radius of the intrarectal balloon segment and the third radius of the transanal balloon section is equal to a third difference, and further wherein the second difference is less than the third difference.

128. The method according to claim 126, wherein, in case of an increased pressure in the balloon beyond 100 mbar, the difference between the larger radius of the distal terminal balloon section and the reduced radius of the proximally adjacent, tapered balloon section will approximate a constant value.

129. The device according to claim 126, wherein the tapered transanal balloon section being adapted to be placed transanally expands by less than 20%, or by less than 15%, at a pressure increase from 30 to 100 mbar.

130. The method according to claim 125, wherein the intrarectal balloon segment being adapted to be placed intrarectally expands by more than 15%, or by more than 20%, at a pressure increase from 30 to 100 mbar.

131. The method according to claim 125, wherein the balloon is made from polyurethane material, or from a material with the same compliance and elasticity characteristics as polyurethane.

132. The method according to claim 126, wherein the tapered transanal balloon section is configured to terminate flush with the anus.

133. The method according to claim 126, wherein the length of the tapered transanal balloon section is designed such that in case of a placement of the device in a patient, the tapered transanal balloon section shall project over the anus to form a pre-anal segment, wherein the pre-anal segment of the balloon is pre-formed into a conical balloon section having a conical shape with a distal end and a proximal end, wherein a pre-formed diameter at the proximal end of the conical balloon section is 30 to 70% larger, or 40 to 60% larger, than a pre-formed diameter at the distal end of the conical balloon section.

134. The method according to claim 126, wherein the intrarectal balloon segment adapted to be placed intrarectally and the tapered transanal balloon section adapted to be placed transanally are manufactured separately, or are manufactured from different materials and/or with different characteristics, or are manufactured from materials with different material characteristics like different durometers.

135. The method according to claim 126, wherein the inflatable balloon has a waisted shape, comprising the intrarectal balloon segment of the first radius and of generally spherical or discoid shape, a proximal terminal balloon section of a fourth radius and of generally spherical or discoid shape, and, disposed between them, the tapered transanal balloon section having a third radius, with the tapered balloon section of the device being adapted to be placed transanally, and the intrarectal balloon segment being adapted to be placed intrarectally, and the proximal terminal balloon section being adapted to be placed extracorporeally, and wherein the fourth radius is larger than the third radius.

136. The method according to claim 125, wherein the ends of the inflatable balloon, beginning at an inflection point of their longitudinal section, are inwardly invaginated, or inverted, and in the inverted or invaginated state are fixed on an outer jacket surface of the catheter shaft.

137. The method as in claim 126, characterized in that an envelope of the evacuated, ready-to-use catheter balloon is placed on the shaft or clings closely thereto in such fashion that the intrarectal and the transanal balloon sections lie in a shaft region between the respective fixations of the upper and lower balloon ends (6, 7), and a preanal balloon portion extends in the proximal direction in a pocket-like manner over gripping depressions (12).

138. The method as in claim 125, characterized in that gripping depressions (12) are disposed on the shaft in direct proximal adjacency to a rear balloon fixation line (14).

139. The method as in claim 125, characterized in that the catheter shaft has a waist (23) in a region of its transanal placement inside the anal canal.

140. The method as in claim 125, characterized in that inserted in a duct (25) of the catheter shaft, or integrated into a tubing line, which connects the catheter shaft with a fluid containing containment, is a non-return element that prevents any retrograde backflow of fluid directed away from the patient through the catheter.

141. The method as in claim 140, characterized in that the non-return element in the duct (25) is a thin-walled tube element having a wall thickness of 5 to 15 micrometers, and having the diameter of the duct (25), wherein the distal end of the tube lies freely in the duct (25) over a length of approximately 5 to 10 mm and its proximal end is sealingly connected to the inner wall of the duct (25), thereby ensuring that the tube element opens in the presence of a flow of medium through the duct toward the tip and the tube element closes by collapsing in the presence of flow in the opposite direction.

142. The method as in claim 125, characterized in that the catheter is an inflow catheter and is connected by a fixed tube connection (32) to a container (33) for the irrigation fluid as a ready-to-use, disposal product.

143. The method as in claim 142, whereby small volumes of fluid as e.g. 5 to 15 ml can be squeezed out from the container (33) into the rectum of a patient, as a micro-enema.

144. The method as in claim 142, whereby a diameter of an irrigation canal of the device is reduced in its dimension to a volume of equal or less than 1 ml, or to a volume of less than 0.5 ml, so as to minimize the volume loss inside this volume.

145. The method as in claim 125, comprising a disposable catheter, characterized in that the disposable catheter is connectable via a filling conduit (34) to a filling device (27), or to a filling device (27) which is implemented as a manually operable pump balloon with a manometer indicating a balloon filling pressure.

146. The method as in claim 125, comprising a manometer indicating a balloon filling pressure, characterized in that the manometer comprises a scale on which filling pressure ranges required for the use of the catheter are specified by suitable markings.

147. The method as in claim 125, characterized by a valve element that limits a balloon pressure and is disposed in or on a reusable filling device (27) or is integrated into a balloon filling conduit (34) of the catheter or into a catheter-bag unit.

148. The method as in claim 125, further comprising a user-adjustable decompression mechanism (37), that when being released, opens the balloon in a pressurized state permanently to an ambient surrounding, once a certain pressure inside the balloon has been reached or exceeded, whereby a pressure increase inside the balloon is configured to be caused by a peristaltic contraction of the colo-rectal bowel segment, due to the released coordinated defecation reflex.

149. The method according to claim 126, wherein, in an inflated, but unpressurized state of the balloon, the difference between the first radius of the intrarectal balloon segment and the third radius of the tapered transanal balloon section is equal to a second difference, and further wherein the second difference is greater than a the distance between a the distal front side of the catheter and a the distal fixation point of the balloon to the catheter shaft; wherein the catheter balloon or at least the intrarectal balloon segment is pre-formed into a working dimension associated with a filling state, characterized by an initial fill volume or an initial filling pressure, in which the filling pressure required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex by generating a reflex-triggering stimulus of an intensity which can, determined by the user, substantially exceed the intensity achievable by an irrigation with fluid media; wherein the catheter balloon or the intrarectal balloon segment has a compliance reserve; wherein the catheter balloon or the intrarectal balloon segment of the catheter balloon distends elastically from the pre-formed working dimension up to a final dimension; and wherein further distention of the catheter balloon or the intrarectal balloon segment of the catheter balloon is not possible once the catheter balloon or intrarectal balloon segment has distended to its final dimension, and no further dimensional expansion is allowed, in order to avoid uncontrolled dilation and trauma.

150. The method according to claim 125, wherein the catheter balloon or at least the intrarectal balloon segment is pre-formed beyond a working dimension associated with a filling state characterized by an initial fill volume or an initial filling pressure, in which the filling pressure in the catheter balloon required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex by generating a reflex-triggering stimulus of an intensity which can, determined by the user, substantially exceed the intensity achievable by an irrigation with fluid media; and wherein the catheter balloon or the intrarectal balloon segment is made of a non-volume-expandable, non-elastic material, such that no further dimensional expansion is allowed beyond a final dimension to which the catheter balloon or the intrarectal balloon segment is pre-formed, in order to avoid uncontrolled dilation and trauma.

151. The method according to claim 126, wherein the catheter balloon or at least the intrarectal balloon segment is pre-formed into a working dimension associated with a filling state, characterized by an initial fill volume or an initial filling pressure, in which the filling pressure required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex; wherein the catheter balloon or the intrarectal balloon segment has a compliance reserve; wherein the catheter balloon or the intrarectal balloon segment of the catheter balloon distends elastically; wherein the transanal balloon segment expands by less than 20%, at a pressure increase from 30 to 100 mbar, and wherein gripping depressions (12) are disposed on the catheter shaft in direct proximal adjacency to a rear balloon fixation line (14).

152. The method according to claim 125, wherein the catheter balloon or at least the intrarectal balloon segment is pre-formed into a working dimension associated with a filling state, characterized by an initial fill volume or an initial filling pressure, in which the filling pressure required for anchoring the catheter within the rectum or colon of the patient is approximately the same as the pressure prevailing in the rectum or abdomen; wherein the catheter balloon is configured such that an increase above the initial fill volume or filling pressure causes a gradual, user-controllable expansion of the catheter balloon such that at least a portion of the catheter balloon engages and expands a side wall of the rectum or colon of the patient, whereby to trigger a coordinated defecation reflex; wherein the catheter balloon or the intrarectal balloon segment has a compliance reserve; wherein the catheter balloon or the intrarectal balloon segment of the catheter balloon distends elastically from the pre-formed working dimension up to a final dimension and is designed to expand by more than 15%, at a pressure increase from 30 to 100 mbar up to a final dimension, where distention comes to a rest and no further dimensional expansion is allowed, in order to avoid uncontrolled dilation and trauma,
